(19) United States
(12) Patent Application Publication
(10) Pub. No.: US 2022/0406400 A1
(43) Pub. Date: Dec. 22, 2022

(54) Systems and Methods to Identify Metabolic Subphenotypes and Uses Thereof

(71) Applicant: The Board of Trustees of the Leland Stanford Junior University, Stanford, CA (US)

(72) Inventors: Ahmed Metwally, Burlingame, CA (US); Michael P. Snyder, Stanford, CA (US); Tracey L. McLaughlin, Menlo Park, CA (US); Dalia Perelman, Stanford, CA (US)

(73) Assignee: The Board of Trustees of the Leland Stanford Junior University, Stanford, CA (US)

(21) Appl. No.: 17/807,619

(22) Filed: Jun. 17, 2022

Related U.S. Application Data
(60) Provisional application No. 63/212,035, filed on Jun. 17, 2021.

(51) Int. Cl. G16B 5/00; G16B 40/20; A61K 31/426; G16H 50/20

(52) U.S. Cl. CPC G16B 5/00; G16B 40/20; A61K 31/426; G16H 50/20

(57) ABSTRACT

Systems and methods to assess metabolic dysregulation are described. Metabolic dysregulation refers to elevated glycemia or insulin resistance. The systems and methods assess metabolic dysregulation by determining which subphenotypes or underlying pathologies are contributing to the metabolic dysregulation. In some instances, a trained computational model utilizes an individual's glucose time series curve to determine the contribution of various metabolic dysregulation subphenotypes to the individual's metabolic dysregulation. Various applications or treatments can be performed based on the determination of metabolic dysregulation subphenotypes.

### CROSS REFERENCE TO RELATED APPLICATIONS

This application claims priority to U.S. Provisional Application Ser. No. 63/212,035 entitled “Systems and Methods to Identify Metabolic Subphenotypes and Uses Thereof,” filed Jun. 17, 2021, which is herein incorporated by reference in its entirety.

### FIELD OF THE DISCLOSURE

The present disclosure relates to diabetes, more particularly, methods and systems to identify and/or treat metabolic subphenotypes that contribute to hyperglycemia associated with Type 2 diabetes.

### BACKGROUND OF THE DISCLOSURE

Type 2 diabetes (T2D) and prediabetes affects over 122 million US adults. It is a heterogeneous condition and the product of complex physiology in which metabolic dysfunction in multiple distinct pathways can contribute to hyperglycemia. Current methods to diagnose diabetes include an oral glucose tolerance test (OGTT) involving fasting and a 2-hour glucose monitoring (after 75 g oral glucose load). However, OGTTs are very crude measures of hyperglycemia, given the inter-individual variability in glucose peak timing and area under the curve and shape of the curve. Furthermore, fasting glucose might be elevated but total glycemic exposure lower, which is also not captured by the current testing methods. Compounding the issue, is the current standard of care, which simply diagnoses diabetes, then treats all patients similarly with a limited array of medications, along with lifestyle changes including weight loss, healthier diet, and exercise. Such blanket approaches fail to appreciate or capture underlying metabolic differences leading to hyperglycemia and/or T2D. As such, there is a need in the art to understand these underlying metabolic differences to effectuate better treatments for an individual.

### SUMMARY OF THE DISCLOSURE

Various embodiments are directed towards systems and methods for assessing metabolic health of an individual. In various embodiments, underlying pathologies of metabolic dysregulation are determined utilizing a glucose time series curve. In various embodiments, the data of the glucose time series curve is utilized in a trained computational model to predict an indicator of one or more underlying pathologies of metabolic dysregulation. In various embodiments, an underlying pathology is one of: muscular insulin resistance, hepatic insulin resistance, beta-cell dysfunction, or impaired incretin effect.

In an embodiment, a method assesses an underlying pathology of metabolic dysregulation in an individual. The method obtains data results of a glucose response curve that is generated from an individual. The method generates features from the data results of the glucose response curve. The method assesses, utilizing a trained computational model, an underlying pathology of metabolic dysregulation. The trained computational model is trained to predict an indicator of the underlying pathology.

In an embodiment, a method stratifies a prediabetic individual based on an underlying pathology of metabolic dysregulation. The method obtains data results of a glucose response curve that is generated from a prediabetic individual. The method generates features from the data results of the glucose response curve. The method assesses, utilizing a trained computational model, an underlying pathology of metabolic dysregulation. The trained computational model is trained to predict an indicator of the underlying pathology.

### BRIEF DESCRIPTION OF THE DRAWINGS

FIG. 1 provides a flow chart of a method to determine one or more underlying pathophysiologies of metabolic dysregulation from the glucose time series curve in accordance with various embodiments.

FIG. 2 provides a flow chart of a method to assess one or more underlying pathologies on metabolic dysregulation in accordance with various embodiments.

FIG. 3 provides a schematic of a processing system in accordance with various embodiments.

FIG. 4 provides a schematic of a study design to interpret metabolic subphenotypes via a machine learning model that utilizes glucose time-series data in accordance with various embodiments.

FIG. 5 provides Table 1 that describes participants characteristics that were involved in the study, utilized in accordance with various embodiments.

FIG. 6 provides time course glucose curves of the participants acquired via a frequently sampled oral glucose tolerance test (OGTT), generated and utilized in accordance with various embodiments. Time=0 on the x-axis represents the time when participants start drinking the glucose drink. The vertical black dashed line represents the time at 120 minutes where the glucose level is measured for clinical diagnosis of diabetes. Glucose value above 200 mg/dL (dashed line) represents a diabetes status, glucose value below 140 mg/dL represents a normal status, glucose value between 140-200 mg/dL represents a prediabetes status, and glucose value below 70 mg/dL represents a hypoglycemia status.

FIG. 7 provides an illustration detailing the participants' metabolic measures of muscle IR, beta cell function, incretin effect, and hepatic IR, sorted by HbA1c levels, utilized in accordance with various embodiments.

FIG. 8 provides a heatmap of participants' insulin secretion rate measured during OGTT normalized by SSPG used to infer beta-cell function in accordance with various embodiments.

FIG. 9 provides glucose time series data of the participants that shows concordance analysis of plasma glucose concentration during OGTT and IIGI, utilized in accordance with various embodiments.

FIG. 10 provides C-peptide concentration time series data of the participants gathered during OGTT and IIGI analysis, utilized in accordance with various embodiments.

FIG. 11 provides GLP-1 concentration time series data of the participants gathered during OGTT and IIGI analysis, utilized in accordance with various embodiments.

FIG. 12 provides GIP concentration time series data of the participants gathered during OGTT and IIGI analysis, utilized in accordance with various embodiments.

FIG. 13 provides glucagon concentration time series data of the participants gathered during OGTT and IIGI analysis, utilized in accordance with various embodiments.

FIG. 14 provides insulin concentration time series data of the participants gathered during OGTT and IIGI analysis, utilized in accordance with various embodiments.

FIG. 15 provides a heatmap showing the standardized deviance score of muscle IR, hepatic IR, beta cell function, and incretin effect for each participant, utilized in accordance with various embodiments. Beta-cell function and incretin effect values have been reversed so that higher positive values represent a greater abnormality. High positive deviance (darker shading) indicates greater deviance from study population average in the abnormal direction (e.g., higher IR or lower incretin effect), while high negative deviance (lighter shading) indicates a healthier metabolic phenotype than the average population. For each participant, the metabolic subphenotype with the highest deviance represents the dominant metabolic subphenotype.

FIG. 16 provides a heatmap schematic showing pairwise correlation network showing the association between the standardized deviance of the four metabolic subphenotypes, utilized in accordance with various embodiments. Pearson correlation coefficient (−1<r<1) was used to determine the strength of each relationship, where r=1 means strong positive correlation, and r=−1 means strong negative correlation. The asterisk symbol on edges means that the correlation is statistically significant (p-value <0.05) between the corresponding two metabolic subphenotypes.

FIG. 17 provides a bar graph depicting T2D polygenic risk score (PRS_{T2D}) for the participants, sorted in descending order and color by the glycemic status measured by HbA1c, utilized in accordance with various embodiments.

FIG. 18 provides a schematic of machine learning framework for predicting metabolic subphenotype using OGTT glucose time series in accordance with various embodiments.

FIG. 19 provides a heatmap schematic depicting a relationship between OGTT glucose time series features and metabolic subphenotypes in accordance with various embodiments. Pearson correlation coefficient (−1<r<1) was used to determine the strength of each relationship, where r=1 “dark red” means strong positive correlation, and r=−1 “dark blue” means strong negative correlation. The “x” in some of the heatmap cells means that the correlation is statistically significant (p-value <0.05) between the curve feature and the corresponding metabolic subphenotype. At the bottom of the figure there is an illustration of some of the glucose curve features. Gₜ denotes plasma glucose level at time t, AUC denotes area under the curve, iAUC denotes incremental area under the curve, pAUC denotes positive area under the curve, nAUC denotes negative area under the curve, and CV denotes coefficient of variation, peak glucose level (G_Peak), length of the glucose time series (CurveSize), time from baseline to peak value (T_baseline2peak), slope between baseline to the peak glucose level (S_baseline2peak), and slope between glucose values at the peak and at the end (at t=180 min) (S_peak2end).

FIGS. 20 to 23 provide principal component analysis of muscle IR (FIG. 20), beta cell dysfunction (FIG. 21), incretin effect (FIG. 22), and hepatic IR (FIG. 23), generated and utilized in accordance with various embodiments.

FIG. 24 is provides calculated HOMA-B, HOMA-IR, and Matsuda index of the participants, utilized in accordance with various embodiments.

FIG. 25 provides a data bar graph depicting the prediction ability of various features within machine learning models, generated in accordance with various embodiments. In total, 8 feature sets were evaluated for each metabolic subphenotype; two sets of features were obtained from OGTT glucose curve (OGTT_G_Features and OGTT_G_ReducedRep), and four measures in current use including Demographics (age, sex, BMI, ethnicity, and participant family history for T2D), Demographics+Lab (demographic variables plus A1C, and FPG), HOMA-B (a surrogate marker for beta-cell function), HOMA-IR and Matsuda Index (both are surrogate markers for muscle insulin resistance), and Incretins (total GIP and GLP-1 concentrations at OGTT_2h, which are optimized surrogate marker for incretin effect). Six classifiers were trained on the training set and the y-axis represents the auROC of the best performing classifier on the test set, for each metabolic subphenotype and for each feature set (FIG. 26 highlights the best performing classifier). Statistical significance is performed between the measure of auROCs among all tested features and OGTT_G_ReducedRep using the Wilcoxon Rank Sum Test.

FIG. 26 provides a schematic showing the best performing classifier for predicting each metabolic subophenotype using separate feature sets, in accordance with various embodiments.

FIG. 27 provides comparison of incretin effect and GLP-1 concentration, generated in accordance with various embodiments.

FIG. 28 provides comparison of incretin effect and GIP concentration, generated in accordance with various embodiments.

FIG. 29 provides comparison of machine learning prediction of muscle IR with measured and calculated SSPG, prediction of beta cell function with measured and calculated DI, prediction of incretin effect with measured and calculated IE %, and predicted hepatic IR with measured and calculated hepatic IR index, generated in accordance with various embodiments.

### DETAILED DESCRIPTION OF THE DISCLOSURE

Turning now to the drawings and data, systems and methods to assess metabolic health and pathoological subphenotypes thereof are described in accordance with the various embodiments of the description. In several embodiments, an individual is asses for metabolic dysregulation and the subphenotypes that contribute to those phenotypes. Herein, metabolic dysregulation refers to glycemia, insulin resistance, type II diabetes, and/or prediabetes. A subphenotype of metabolic dysregulation is any underlying pathology that contributes to the manifestation and/or progression of metabolic dysregulation. Subphenotypes include (but are not limited to) muscular insulin resistance, hepatic insulin resistance, beta-cell dysfunction, and impaired incretin effect.

Many embodiments are direct to determine a subphenotype of metabolic dysregulation via a glucose time series curve, which can be generated via an oral glucose tolerance test (OGTT), continuous glucose monitoring (CGM), or other similar assessments. Compared to many other glycemia related tests, OGTT is a fairly easy clinical assessment that is performed rather routinely. Several other assessments, such as assessments for insulin resistance, beta cell function, and incretin defect are much more difficult to perform, but these assessments provide an understanding of which underlying pathologies are contributing to the development of elevated glycemia. Accordingly, it would be ideal to utilize glucose time course series to determine any underlying pathologies.

Based on the methods, data, and results described herein, it is now known that a glucose time series curve can be analyzed to determine which subphenotypes contribute to metabolic dysregulation. And in some particular embodiments, the most prominent subphenotype contributing to metabolic dysregulation can be determined. In several embodiments, a glucose time series curve is analyzed via trained computational model that determines the which subphenotypes (including the most prominent subphenotypes) contribute to metabolic dysregulation.

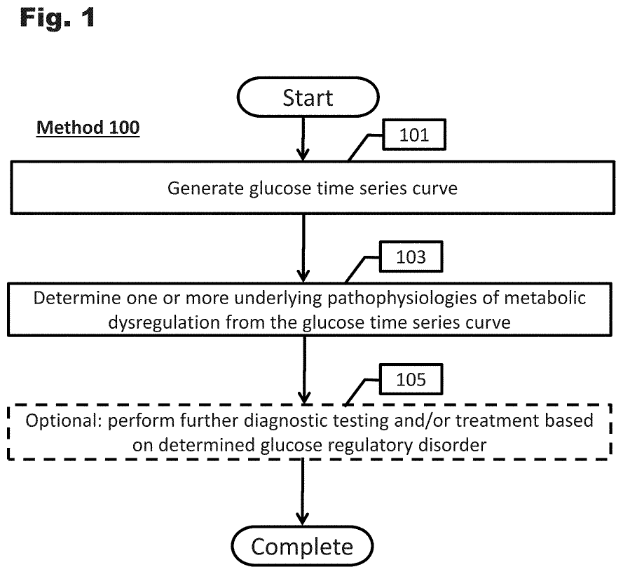

An overview method of assessing metabolic dysregulation is provided in FIG. 1. Method 100 can begin by generating (101) glucose time series curve of an individual. A glucose time series curve is the glycemic level of glucose over time. A glucose time series curve can be generated by extracting blood at several time points or by utilizing a continuous glucose monitor (e.g., Dexcom G6 CGM system). In several embodiments, a glucose time series curve is measured after a glucose load, meal, or any other consumption glucose. Often, and in accordance with many embodiments, a glucose load of a particular amount is administered and blood glucose levels are measured at distinct time intervals after administration. In some embodiments, a glucose time series curve is captured during an oral glucose tolerance test (or similar assessment).

After generating a glucose time series curve, one or more underlying pathologies of metabolic dysregulation is determined (103). Based on recent experimentation and generated data, it is now known that a glucose time series curve can be utilized to assess several underlying pathologies (also referred to as subphenotypes). These underlying pathologies include (but are not limited to) muscular insulin resistance, hepatic insulin resistance, beta cell dysfunction, and incretin defect. In some instances, multiple underlying pathologies of metabolic dysregulation are assessed and compared to yield a determination of which underlying pathologies are prominent in the development of metabolic dysregulation. In many embodiments, an underlying pathology of metabolic dysregulation is determined is determined by a computational model.

Method 100 optionally performs (103) further diagnostic testing and/or treatment based on determined glucose regulatory disorder. Because OGTT and CGM measurements are relatively easy to obtain, these measurements can be utilized as an initial assessment to determine the subphenotypes of elevated glycemia. If the initial assessment suggests one or more subphenotypes are a prominent underlying pathology of prediabetes or type 2 diabetes, then a further clinical test can be performed to confirm such assessment. Furthermore, various treatments can be administered to treat the one or more underlying phenotypes.

### Computational Models to Predict Underlying Pathology Indicator of Metabolic Dysregulation

Several embodiments are directed towards utilizing a computational model to predict an underlying pathology indicator on metabolic dysregulation. Generally, a computational model can be trained to predict whether an individual has an underlying pathophysiological condition that is causing or creating a risk of elevated glycemia as determined by the individual's glucose time series curve. Any subphenotype of metabolic dysregulations can be assessed. In various embodiments, one or more for the following underlying pathologies (or subphenotypes) are assessed: muscular insulin resistance, hepatic insulin resistance, beta-cell dysfunction, and impaired incretin effect.

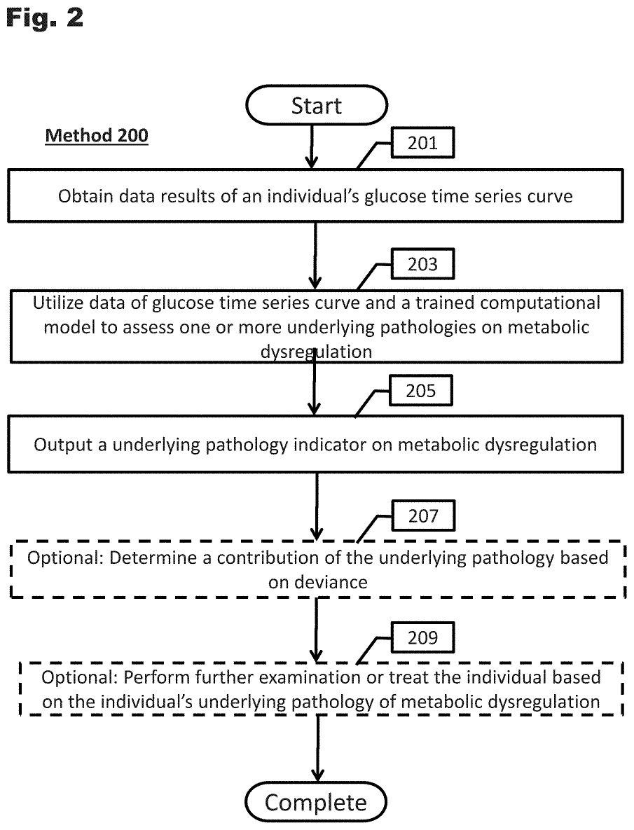

Provided in FIG. 2 is an exemplary method for predicting an indicator of an underlying pathology on metabolic dysregulation. Method 200 begins with obtaining (201) data results of an individual's glucose time series curve. A glucose time series curve is collection of blood glucose levels over a period of time in response to a glucose load, a meal, or any other consumption glucose. In some embodiments, a glucose time series curve is generated via an OGTT. In some embodiments, a glucose time series curve is generated via a CGM.

A trained computational is also obtained. The trained computational model has been trained on glucose time series curves to predict an indicator of underlying pathology on metabolic dysregulation. Multiple trained computational models can be utilized, one for each subphenotype to be assessed. Any type of predictive computational model and architecture can be utilized. In several embodiments, the predictive computational model is a classifier. Examples of predictive computational classifiers that can be utilized include (but are not limited to) Gaussian process classifier (GPC), support vector machine with a radial basis function kernel (SVM-RBF), support vector machine with a linear kernel (SVM-linear), logistic regression with L1 regularization (LR-L1), and logistic regression with L2 regularization (LR-L2). In some embodiments, a computational model is trained to predict an indicator of steady state plasma glucose (SSPG) as an indication of muscular IR. In some embodiments, a computational model is trained to predict an indicator of disposition index (DI) as an indication of beta cell function. In some embodiments, a computational model is trained to predict an indicator of incretin effect percent (IE %) as an indication of impaired incretin effect. In some embodiments, a computational model is trained to an indicator of hepatic IR index as an indication of hepatic IR. For more on training computational models, see Exemplary Embodiments for exemplary methods.

The data results of an individual's glucose time series curve are utilized (203) as features in the trained computational model to assess one or more underlying pathologies of metabolic dysregulation. Any technique to generate features from glucose time series curve data can be utilized.

In some embodiments, features are extracted from the individual's glucose time series curve. Extracted features can include (but are not limited to) glucose level at 0 seconds (G₀), glucose level at 60 minutes (G₆₀), glucose level at 120 minutes (Gin), glucose level at 180 minutes (Gin), peak glucose level (G_Peak), length of the glucose time series (CurveSize), area under the curve (AUC), positive area under the curve (pAUC), negative area under the curve (nAUC), incremental area under the curve (iAUC), coefficient of variation (CV), time from baseline to peak value (T_baseline2peak), slope between baseline to the peak glucose level (S_baseline2peak), and slope between glucose values at the peak and at the end (at t=180 min) (S_peak2end).

In some embodiments, a reduced representation of the individual's glucose time series curve is utilized. To get a reduced representation, a glucose time series curve can be smoothed and Z-normalized, and then the one or more top principal components can be extracted via eigen-decomposition of the covariance matrix.

Any number of top principal components can be extracted and utilized. In some embodiments, the top principal component is extracted and utilized. In some embodiments, the top principal component is extracted and utilized. In some embodiments, the top two principal components are extracted and utilized. In some embodiments, the top three principal components are extracted and utilized. In some embodiments, the top four principal components are extracted and utilized. In some embodiments, the top five principal components are extracted and utilized. In some embodiments, the top six principal components are extracted and utilized. In some embodiments, the top seven principal components are extracted and utilized. In some embodiments, the top eight principal components are extracted and utilized. In some embodiments, the top nine principal components are extracted and utilized. In some embodiments, the top ten principal components are extracted and utilized.

The extracted glucose time course series features are utilized in a computational predictive model to yield an assessment of an underlying pathology. Examples of results that can be yielded from a trained computational model include a score or classification indicative of: muscular IR, beta cell dysfunction, impaired incretin effects, or hepatic IR. Method 200 outputs (205) the underlying pathology indicator of metabolic dysregulation. In various embodiments, one or more computational models are utilized to yield a score or classification indicative of one or more of: SSPG, DI, IE %, and hepatic IR index. In various embodiments, one or more computational models are utilized to yield a score or classification indicative of one or more of: muscular IR, beta cell dysfunction, impaired incretin effects, and hepatic IR. In some embodiments, a computational model framework yields a score or classification indicative of the following: SSPG, DI, IE %, and hepatic IR index. In some embodiments, a computational model framework yields a score or classification indicative of the following: muscular IR, beta cell dysfunction, impaired incretin effects, and hepatic IR.

Method 200 optionally determines (207) a contribution of the underlying pathology on metabolic dysregulation based on deviance. In some embodiments, the dominant subphenotype (i.e., top contributor) is determined. Deviance of underlying pathology from a healthy pathology can be determined by any appropriate means. In several embodiments, deviance from a healthy pathology is determined by the deviation from an average underlying pathology score from a collection of individuals. In some embodiments, the collection of individuals has a similar overall metabolic assessment. For example, the collection of individuals may all have similar diabetic diagnosis. In various embodiments, the collection of individuals is healthy, prediabetic, or diabetic, as determined by HbA1C levels. An example of computing deviance to identify contribution of an underlying pathology and/or the dominant underlying pathology is provided within the Exemplary Embodiments.

Furthermore, based on an individual's indicated metabolic health, the individual is optionally further examined and/or treated (209) based on the individual's underlying pathology of metabolic dysregulation. In several embodiments, the individual is further assessed for metabolic dysregulation, which can include performing a clinical diagnostic and/or calculation to determine one or more of: SSPG, DI, IE %, and hepatic IR index. In many embodiments, the clinical diagnostic and/or calculation is performed to confirm the prediction of the computational model. In several embodiments, an individual is provided with a treatment plan tailored to the contribution of underlying pathology. Further discussion of treatments is described in detail below, which may include various dietary adjustments, medications, and dietary supplements.

Many embodiments are directed to better stratifying various individual by determining the contribution of one or more underlying pathologies. Accordingly, an individual that is classified as diabetic, prediabetic, or healthy can be further stratified by this contribution. Stratification of an individual can help determine an appropriate course of action, especially for an individual that is classified as prediabetic.

A method for stratification of an individual can be as follows:
Determine or have provided an overall diagnostic of metabolic health
Determine a contribution of an underlying pathology
Based on the contribution of an underlying pathology, determine an appropriate course of action

An overall diagnostic of metabolic health can be classification as diabetic, prediabetic, or healthy. This classification can be performed by any appropriate means. In some embodiments, HbA1c levels or fasting glucose levels are used to classify overall metabolic health.

Determination of a contribution of an underlying pathology can be performed utilizing a method, such as (for example) Method 200 of FIG. 2. This can provide an indication of one of the following: whether an individual is insulin resistant or insulin sensitive; whether an individual has beta cell dysfunction; or whether an individual has impaired incretin effect. Accordingly, an individual can be further stratified based on the underlying pathology contribution.

An appropriate course of action may be determined. As is described below, various treatments and life style changes may be administered to an individual based on their underlying pathology stratification. For example, a prediabetic individual may be further stratified as insulin resistant or insulin sensitive. In some embodiments, insulin resistant individuals would be administered a more aggressive treatment that incudes medications along with dietary alterations and increased exercise; whereas insulin sensitive individuals would be administered dietary alterations and increased exercise without medication.

While specific examples of methods for predicting an individual's underlying pathology effect on metabolic dysregulation are described above, one of ordinary skill in the art can appreciate that various steps of the process can be performed in different orders and that certain steps may be optional according to some embodiments of the disclosure. As such, it should be clear that the various steps of the process could be used as appropriate to the requirements of specific applications. Furthermore, any of a variety of processes for predicting an individual's underlying pathology as appropriate to the requirements of a given application can be utilized in accordance with various embodiments of the disclosure.

### Computational Processing System

A computational processing system to evaluate subphenotypes of metabolic dysregulation in accordance with various embodiments of the disclosure typically utilizes a processing system including one or more of a CPU, GPU and/or other processing engine. In some embodiments, the computational processing system is housed within a computing device. In certain embodiments, the computational processing system is implemented as a software application on a computing device such as (but not limited to) mobile phone, a tablet computer, and/or portable computer. In some embodiments, the computational processing unit is housed with or in direct communication with a glucose monitor, such as (for example) a continuous glucose monitor.

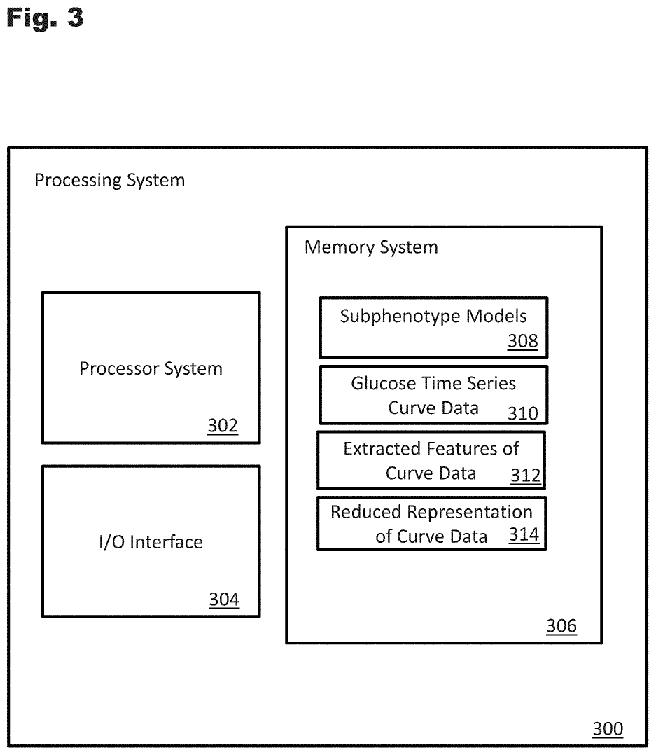

A computational processing system in accordance with various embodiments of the disclosure is illustrated in FIG. 3. The computational processing system 300 includes a processor system 302, an I/O interface 304, and a memory system 306. As can readily be appreciated, the processor system 302, I/O interface 304, and memory system 306 can be implemented using any of a variety of components appropriate to the requirements of specific applications including (but not limited to) CPUs, GPUs, ISPs, DSPs, wireless modems (e.g., WiFi, cellular, Bluetooth modems), serial interfaces, volatile memory (e.g., DRAM) and/or non-volatile memory (e.g., SRAM, and/or NAND Flash). In the illustrated embodiment, the memory system is capable of storing various computational models and data, each of which are optional and/or combinable in any fashion. Computational models include subphenotype models 308. A subphenotype model 308 can predict the underlying pathology of a metabolic dysregulation of an individual based on glucose time series curve data, which can be entered via the I/O interface or gathered via a connection (e.g., WiFi, cellular Bluetooth). Subphenotype models include any model for assessing a subphenotype of elevated glycemia, including (but not limited to) muscular IR, beta cell dysregulation, incretin defect, and hepatic IR. Computational models 308 can be classifier models, such as (for example) as discussed in reference to FIG. 2. The various model applications can be downloaded and/or stored in non-volatile memory. Glucose time series curve data 310 can optionally be stored in non-volatile memory, which can be utilized in any of the applications and/or models or used to keep a data log. Extracted features of curve data 312 and reduced representations of curve data 314 can be generated from the glucose time series curve data 310 and can optionally be stored in non-volatile memory and utilized with the subphenotype models 308. When executed, the various applications and models are each capable of configuring the processing system to implement computational processes including (but not limited to) the computational processes described above and/or combinations and/or modified versions of the computational processes described above.

While specific computational processing systems are described above with reference to FIG. 3, it should be readily appreciated that computational processes and/or other processes utilized in the provision of subphenotype assessment and metabolic health evaluation in accordance with various embodiments of the disclosure can be implemented on any of a variety of processing devices including combinations of processing devices. Accordingly, computational devices in accordance with embodiments of the disclosure should be understood as not limited to specific computational processing systems. Computational devices can be implemented using any of the combinations of systems described herein and/or modified versions of the systems described herein to perform the processes, combinations of processes, and/or modified versions of the processes described herein.

### Applications and Treatments Related to Metabolic Regulation

Various embodiments are directed to applications and treatments related to metabolic regulation and more particularly to subphenotypes of metabolic dysregulation. As described herein, a subject may have their subphenotypes of metabolic dysregulation indicated by various methods. Based on a subject's subphenotype indication, the subject can be treated with various medications, dietary supplements, dietary alterations, and physical exercise regimens.

A number of embodiments of the disclosure are directed towards determining an underlying mechanistic indication of an individual's pathology of a metabolic dysregulation and treating the individual accordingly. In various embodiments, a computational assessment based on glucose time series curve data is performed on an individual that illuminate a pathological indicator of metabolic dysregulation. In some embodiments, a dominant underlying pathology is determined. In some embodiments, individuals are treated with medicaments and/or supplements that specifically target an indicated underlying pathology.

In accordance with American Diabetes Association (ADA) “Standard of Medical Care in Diabetes,” current practices of treating prediabetes and type 2 diabetes do not utilize indicators of underlying pathology, but instead use a trial-and-error approach (see American Diabetes Association, Diabetes Care, 41 (Supplement 1) S73-S85 (January 2018), the disclosure of which is incorporated herein by reference). The ADA recommends beginning treatment with Metformin and further may include treatment with insulin for newly diagnosed patients meeting certain criteria. If the initial mono or dual treatment does not work, then an additional antihyperglycemic agent is added. The ADA further recommends other treatments based on response to the initial treatments, but none of the recommended treatments are actually based on the underlying pathology of glycemic dysregulation.

In various embodiments, underlying pathologies that can be assessed by methods described herein include (but are not limited to) muscular IR, beta cell dysregulation, incretin defect, hepatic IR, and combinations thereof. Each assessment provides unique information that can be utilized to get an indication of glycemic dysregulation pathology.

Based on an individual's indicated underlying pathology of glycemic dysregulation, the individual is treated. A number of treatments are described throughout. In particular, an individual can be treated with medicaments and supplements directed at the individuals' indicated pathology. In some embodiments, when an individual has been indicated to have beta cell dysregulation, the individual is treated with agents that improve insulin secretion, which may include DPP-4 inhibitors (e.g., alogliptin, linagliptin, saxagliptin, sitagliptin, vildagliptin, gemigliptin, anagliptin, teneligliptin, trelagliptin, omarigliptin, evogliptin, gosogliptin, dutogliptin, berberine), sulfonylureas (e.g., glimepiride, gliclazide, glyburide, chlorpropamide, tolazamide, tolbutamide, acetohexamide, carbutamide, metahexamide, glycyclamide, glibornuride, glipizide, gliquidone, glisoxepide, glyclopyramide), GLP-1 receptor agonists (e.g., glucagon-like peptide 1, gastric inhibitory peptide, albiglutide, dulaglutide, exenatide, liraglutide, lixisenatide, semaglutide), and Panax ginseng. In various embodiments, when an individual has been indicated to have muscular insulin resistance, the individual is treated with agents that improve insulin sensitivity, which may include thiazolidinedione (e.g., rosiglitazone, pioglitazone, lobeglitazone). In some embodiments, when an individual has been indicated to have hepatic insulin resistance, the individual can be treated with agents that decrease hepatic glucose production, which may include biguanides (e.g., metformin) and thiazolidinediones (e.g., rosiglitazone, pioglitazone, lobeglitazone). In some embodiments, when an individual has been indicated to have an impaired incretin effect, the individual can be treated with GLP-1 receptor agonists (e.g., glucagon-like peptide 1, gastric inhibitory peptide, albiglutide, dulaglutide, exenatide, liraglutide, lixisenatide, semaglutide) and DPP-4 inhibitors (e.g., alogliptin, linagliptin, saxagliptin, sitagliptin, vildagliptin, gemigliptin, anagliptin, teneligliptin, trelagliptin, omarigliptin, evogliptin, gosogliptin, dutogliptin, berberine).

### Medications and Supplements

Several embodiments are directed to the use of medications and/or dietary supplements to treat a subject to mitigate and/or prevent metabolic dysregulation, including (but not limited to) insulin resistance and/or hyperglycemia. In some embodiments, medications and/or dietary supplements are administered in a therapeutically effective amount as part of a course of treatment. As used in this context, to “treat” means to ameliorate or prophylactically prevent at least one symptom of the disorder to be treated or to provide a beneficial physiological effect. For example, one such amelioration of a symptom could be reduction of insulin resistance and one such prophylactic could be prevention of hyperglycemia. Assessment of glycemic regulation can be performed in many ways, including (but not limited to) assessing subphenotypes as described herein and/or assessing glycemia by insulin suppression test, OGTT, glucose levels, and HbA1c levels. Treatments as described herein can be administered to an individual having an underlying pathology of metabolic dysregulation but otherwise are not experiencing hyperglycemia at levels of type II diabetes. In some embodiments, the individual has underlying pathology of metabolic dysregulation but has healthy to prediabetic levels of glycemia.

A therapeutically effective amount can be an amount sufficient to prevent reduce, ameliorate or eliminate the symptoms of diseases or pathological conditions susceptible to such treatment, such as, for example, diabetes, heart disease, or other diseases that are affected by elevated glycemia. In some embodiments, a therapeutically effective amount is an amount sufficient to reduce an individual's insulin resistance and/or improve an individual's glucose tolerance. In similar embodiments, a therapeutically effective amount is an amount sufficient to reduce a subject's insulin resistance or hyperglycemia result below a certain threshold.

A number of medications are available to treat metabolic dysregulation, such as those used to treat type II Diabetes. Medications include (but are not limited to) insulin, alpha-glucosidase inhibitors (e.g., acarbose, miglitol, voglibose), biguanides (e.g., metformin), dopamine agonists (e.g., bromocriptine), DPP-4 inhibitors (e.g., alogliptin, linagliptin, saxagliptin, sitagliptin, vildagliptin, gemigliptin, anagliptin, teneligliptin, trelagliptin, omarigliptin, evogliptin, gosogliptin, dutogliptin, berberine), GLP-1 receptor agonists (e.g., glucagon-like peptide 1, gastric inhibitory peptide, albiglutide, dulaglutide, exenatide, liraglutide, lixisenatide, semaglutide), meglitinides (e.g., nateglinide, repaglinide), sodium glucose transporter 2 inhibitors (e.g., dapagliflozin, canagliflozin, empagliflozin, ertugliflozin, ipragliflozin, luseogliflozin, sotagliflozin, tofogliflozin), sulfonylureas (e.g., glimepiride, gliclazide, glyburide, chlorpropamide, tolazamide, tolbutamide, acetohexamide, carbutamide, metahexamide, glycyclamide, glibornuride, glipizide, gliquidone, glisoxepide, glyclopyramide), and thiazolidinediones (e.g., rosiglitazone, pioglitazone, lobeglitazone). Furthermore, as described herein, a subject can be treated for insulin resistance with sialylated IgG Fc. Accordingly, an individual may be treated, in accordance with various embodiments, by a single medication or a combination of medications described herein. Furthermore, several embodiments of treatments further incorporate heart disease medications (e.g., aspirin, cholesterol and high blood pressure medications), dietary supplements, dietary alterations, physical exercise, or a combination thereof.

Numerous dietary supplements may also help to treat metabolic dysregulation. Various dietary supplements, such as alpha-lipoic acid, chromium, coenzyme Q10, garlic, hydroxychalcone (cinnamon), magnesium, omega-3 fatty acids, psyllium and vitamin D have been shown to have beneficial effects on individuals having diabetes and cardiac conditions. Thus, embodiments are directed to the use of dietary supplements, included those listed herein, to be used to treat a subject based on a subject's metabolic dysregulation result. A number of embodiments are also directed to combining dietary supplements with medications, dietary alterations, and physical exercise to reduce glycemic variability.

### Diet and Exercise

Numerous embodiments are directed to dietary alteration and exercise treatments. Altering one's lifestyle, including physical activity and diet, has been shown to improve glycemic regulation. Accordingly, in a number of embodiments, an individual is treated by altering their diet and increasing physical activity in response to an insulin resistance assessment result. This is especially true for individuals that have an underlying pathology of metabolic dysregulation but otherwise are not experiencing hyperglycemia at levels of type II diabetes. In some embodiments, the individual has underlying pathology of metabolic dysregulation but has healthy to prediabetic levels of glycemia.

There are various diets that will help different individuals in getting better glycemic control. A number of embodiments are directed to treatments to reduce weight, which has been considered by some to be the best approach to control one's glycemia. There are many programs based on the seminal study for a low-fat diet to prevent diabetes (see Diabetes Prevention Program (DPP) Research Group. Diabetes Care. 2002 25:2165-71, the disclosure of which is herein incorporated by reference). For others, a diet low in refined carbohydrates and sugars will work better. Numerous embodiments take a more personalized approach such that one can utilize continuous glucose monitoring (CGM) results to determine which foods cause glycemic spikes for an individual and devise a diet to limit these particular foods while maintaining appropriate nutrient intake. Numerous embodiments are directed to treating an individual by substituting saturated fats with monounsaturated and polyunsaturated fats to help lower the risk for cardiovascular disease, which would be beneficial for many individuals with underlying pathologies of metabolic dysregulation. Also, embodiments are directed to increasing amounts of fiber in the diet, which would be highly recommended to both help with metabolic regulation and also balance serum lipid levels (cholesterol and triglycerides).

Exercise has a large impact on metabolic regulation. In several embodiments, a treatment would entail a minimum of some minutes of active exercise per week. In some embodiments, treatments would include a minimum of 150 minutes of exercise a week, however, the precise duration of exercise may be dependent on the individual to be treated and their cardiovascular health. It is further noted that cardiovascular exercise is important for the immediate glycemic control and weight training will have a long-term effect by increasing muscle mass, affecting glucose utilization during rest.

In many embodiments, a treatment to help control glucose levels is stress management, as stress increases blood glucose levels. Some proven ways to help control stress include meditation, social support, adequate sleep, journaling, and therapy.

### Exemplary Data

Bioinformatic and biological data support the methods and systems of assessing metabolic healthy and applications thereof. In the description below, exemplary data and exemplary applications describe computational systems and methods that utilize machine learning to predict metabolic subphenotypes from glucose curves. Further provided is description of the relationship between metabolic subphenotypes and glucose dysregulation, which can be utilized as a basis for determining underlying pathologies of diabetes and pre-diabetes and determining treatment options.

### Machine Learning Framework Predicts Metabolic Subphenotypes Using Glucose Time Series

Type 2 diabetes (T2D) is classically defined by measures of glycemia without accounting for the pathophysiologic basis. It is likely that muscle insulin resistance, beta-cell dysfunction, incretin defect, and hepatic insulin resistance are present to varying degrees in individuals predisposed to T2D. The data provided in herein show that these individuals can be classified according to their metabolic subphenotype as determined from their glucose timeseries during oral glucose tolerance test (OGTT). Thirty-two participants underwent gold-standard metabolic tests including frequently-sampled 3-hour OGTT, with c-peptide deconvolution to quantify beta-cell function, steady-state plasma glucose test to measure insulin resistance, isoglycemic intravenous glucose infusion to quantify incretin effect, and hepatic insulin resistance. A machine learning framework was developed to predict metabolic subphenotype of an individual using dynamic features of the glucose time series, or shape of the curve, during the OGTT. Metabolic testing revealed substantial inter-individual heterogeneity in metabolism and the existence of “dominant” metabolic subphenotypes. The features of the glucose time series identified insulin resistance, beta-cell deficiency, and incretin deficiency with auROCs of 94%, 76%, and 75%, respectively, which were superior to currently-used estimates. This capability with a single, relatively practical test sets the stage for targeted lifestyle or pharmacologic therapy to prevent and/or treat T2D or prediabetes.

Importance of Assessing Diabetes Precisely and Issues with Current State of Diagnostics

Type 2 diabetes (T2D) affects over 537 M adults globally. Diabetes and prediabetes are often defined by an elevation in blood glucose, but the underlying physiology is complex and likely to differ between individuals. It is traditionally suggested that individuals who develop T2D have both insulin resistance (IR) and beta-cell dysfunction. However, data on individuals with prediabetes and early T2D shows that they are not always insulin resistant, implicating other physiologic contributors. In addition, forty to fifty percent of the nondiabetic US adult population has IR, which significantly increases the risk for T2D, cardiovascular disease, stroke, hypertension, non-alcoholic liver disease, and cancer. Other contributors to glucose homeostasis include beta-cell function, incretin action, and hepatic glucose regulation. Altered metabolism is present prior to the onset of overt hyperglycemia, and both lifestyle and medical interventions have been shown to prevent conversion to T2D in high-risk individuals.

Given that metabolic dysfunction precedes glucose elevation and earlier detection might prevent onset of diabetes, it is desirable to characterize high-risk individuals by the pathological metabolic defect rather than glucose elevation. Furthermore, multiple approved T2D treatments targeting specific physiologic components of glucose regulation now exist, which also highlights the potential benefit of redefining T2D classification to include individual physiologic differences in glucose dysregulation. In fact, these varied classes of antihyperglycemic therapy have also been proven to prevent progression to T2D in high risk individuals, further supporting the benefit of an updated classification approach that would allow targeted interventions to prevent T2D. If this were possible in the clinical setting, advancements in precision health and medicine, with individualized lifestyle and medical interventions, would likely ensue, both improving health and reducing health care expenditures on less effective non-targeted treatments. There is a growing interest in identifying scalable cost-effective tests which capture the complexity of a phenotype, including those based on partitioned genetic risk scores and wearable devices. It is suggested here that prediabetic and early diabetic state is characterized by significant metabolic heterogeneity between individuals and that identification of metabolic “subphenotypes” is possible. It is further suggested that features of the glucose response to standardized administration of an oral glucose load could identify the specific metabolic abnormalities underlying glucose dysregulation, which, if replicated by continuous glucose monitor-derived curves, would represent a scalable and relatively cost-effective method that would facilitate individualized prevention and treatment of T2D, thereby facilitating precision approaches to health and medicine.

Herein, it is demonstrated that the physiologic basis for glucose dysregulation differs between individuals, with some individuals demonstrating severe IR and others normal insulin sensitivity but marked beta-cell dysfunction, incretin defect, and/or hepatic insulin resistance. These data suggest it is possible to reframe subclassification of prediabetes and possibly early T2D so that targeted treatments can be utilized, even before the onset of overt hyperglycemia. A novel and comprehensive framework using machine learning is described, by which metabolic subphenotypes can be identified using features of the glucose time series extracted during a glucose tolerance test. The accuracy of the classification system is greater than standard glycemia measures, other surrogate markers in current use, including genetic risk score. Together these results provide scientific rationale and feasibility to support metabolic subphenotyping to inform personalized approaches to diabetes prevention and treatment.

### Research Design and Methods

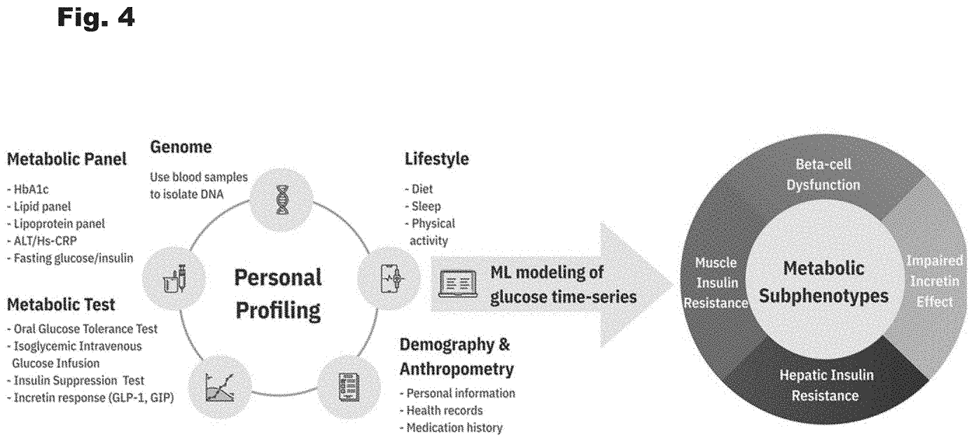

Deep metabolic profiling was performed on 32 participants using gold-standard quantitative tests with the goal of assessing four distinct physiologic processes known to contribute to glucose dysregulation and T2D: muscle insulin resistance, beta-cell dysfunction, impaired incretin action, and hepatic insulin resistance (FIG. 4). All metabolic tests were performed in the Stanford University Clinical Translational Research Unit after an overnight fast. The tests included 1) a standardized frequently-sampled OGTT in which plasma glucose was obtained following a 75 g oral glucose load at 5-15 minute intervals to generate a precise glucose time series used for modeling; 2) an isoglycemic intravenous glucose infusion (IIGI) with measurements of glucose and C-peptide to quantify the incretin response; 3) a modified insulin suppression test measuring steady-state plasma glucose (SSPG) to quantify insulin-mediated glucose disposal, which largely reflects the degree of muscle insulin resistance. Hepatic insulin resistance was calculated using a formula validated against endogenous glucose production measured during euglycemic-hyperinsulinemic clamp. Utilizing these measurements, a model was devloped using features extracted from the OGTT-generated glucose time series. These features included parameters such as glucose peak height, area under the curve (AUC), curve length, etc. The four metabolic subphenotypes were modeled from the glucose time series data using a novel machine learning framework and the performance of each model was then assessed. For comparison to other surrogate markers for metabolism, fasting and post-glucola insulin, calculated HOMA-IR, HOMA-B, Matsuda index24, GLP-1 and GIP total concentrations, and clinical and genetic markers were also measured.

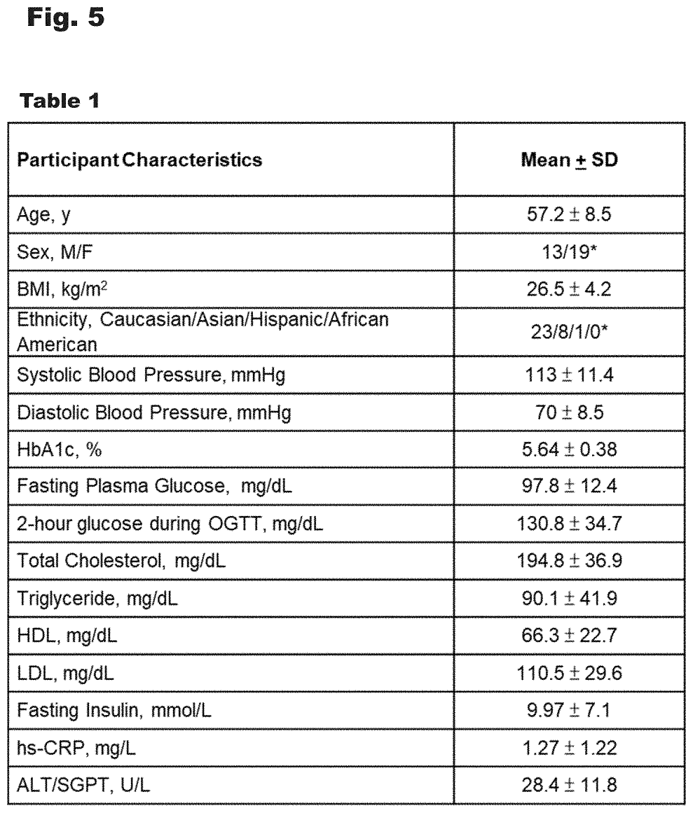

Study participants: The study protocol and clinical investigation were approved by the Stanford University School of Medicine Human Research Protection Office (Institutional Review Board #43883). Participants provided written informed consent. Participants were recruited from the San Francisco Bay Area via locally placed advertisements online, in local newspapers, and during faculty lectures to the community. Participants were screened with medical history and physical examination in the Stanford University Clinical and Translational Research Unit (CTRU), where vital signs, fasting plasma blood glucose, and baseline hematocrit, ALT, and creatinine were obtained. Eligibility criteria included age 30-70 years, BMI 23-40 kg/m2, absence of major organ disease, type 2 diabetes defined by fasting glucose <126 mg/dl (with one exception), uncontrolled hypertension, malignancy, chronic inflammatory conditions, use of any medications known to alter blood glucose or insulin sensitivity, hematocrit <30, creatinine above the normal range, and ALT >two-fold above the upper limit of the normal range. The cohort's characteristics are summarized in Table 1 of FIG. 5. Based on criteria set for by American Diabetes Association, 17 were normoglycemic (HbA1c<5.7% [6.49 mmol/L]), 14 had prediabetes (5.7% [6.49 mmol/L]≤HbA1c≤6.4% [7.61 mmol/L]), and one had T2D (HbA1c>6.4% [7.61 mmol/L]: negative autoantibody tests were obtained).

Quantitative metabolic physiological tests: All tests were performed in the Stanford CTRU after a ten-hour overnight fast.

Frequently-sampled oral glucose tolerance test (OGTT): Dynamic plasma glucose profiles were obtained after administration of a 75 g oral glucose load administered over 5 minutes (Brand Trutol, Manufacturer Fisher, 75 g in a 10 oz serving). Plasma samples were drawn from an antecubital intravenous catheter drawn at 16 timepoints (−10, 0, 10, 15, 20, 30, 40, 50, 60, 75, 90, 105, 120, 135, 150, and 180 min). Glucose level was measured at the 16 timepoints (using the oximetric method), insulin and C-peptide (using the Millipore radioimmunoassay assay), both at 7 timepoints (0, 15, 30, 60, 90, 120, 180 min), GLP-1 (using Millipore ELISA EZGLP1T-36K kit), GIP (using Millipore ELISA EZHGIP-54K kit), Glucagon (using Mercodia ELISA 10-1271-01 kit), all at 4 timepoints (0, 30, 60, 120 min). Insulin, C-peptide, GLP-1, GIP, and Glucagon were measured at the Core Lab for Clinical Studies, Washington University School of Medicine in St. Louis. From the baseline sample (at 0 min), HbA1c, fasting plasma glucose, insulin, triglyceride, and high-density lipoprotein cholesterol levels were determined.

Isoglycemic intravenous glucose infusion (IIGI): IIGI was performed in order to quantify the incretin effect by duplicating the plasma glucose profile during the corresponding OGTT. During an IIGI test, an intravenous catheter was placed in the antecubital vein for administration of continuous dextrose infusion at a rate needed to obtain desired glucose, which for each time point was the glucose levels obtained at the same time point during the OGTT. Blood sampling from a second intravenous catheter duplicated the same time points and assays as described above for the OGTT.

Insulin suppression test (IST): Insulin-mediated glucose disposal was quantified by a modification of the insulin suppression test as originally described and validated (see S. Shen, G. M. Reaven, and J. W. Farquhar J. Clin. Invest. 49, 2151-2160 (1970); M. S. Greenfield, et al., Diabetes 30, 387-392 (1981); the disclosures of which are each incorporated by reference). Briefly, participants were infused for 240 min with octreotide (0.27 μg m⁻² min⁻¹) (to suppress endogenous insulin secretion), insulin (32 mU m⁻² min⁻¹) and glucose (267 mg m² min⁻¹). Blood was drawn at 30 min intervals for monitoring and at 10-min intervals from 210 to 240 min of the infusion to measure plasma glucose and insulin concentrations, and the mean of these four values was used as the steady-state plasma insulin (SSPI) and glucose (SSPG) concentrations for each individual. As SSPI concentrations are similar in all participants during these tests, the SSPG concentration provides a direct measure of the ability of insulin to mediate disposal of an infused glucose load; the higher the SSPG concentration, the more insulin-resistant the individual.

Calculation—Muscle insulin resistance: SSPG was used to indicate muscle insulin resistance since the majority of insulin-mediated glucose disposal occurs in muscle which was determined by taking the mean of the four glucose measurements at minutes 150, 160, 170 and 180 during the IST.

Calculation—Beta-cell function (Disposition Index (DI)): In order to quantify the status of the beta-cell function, first calculated insulin secretion rate using C-peptide deconvolution method, using C-peptide concentrations measured during OGTT tests at 0, 15, and 30 mins. The Insulin SECretion (ISEC) software (R. Hovorka, P. A. Soons, and M. A. Young, Comput. Methods Programs Biomed. 50, 253-264 (1996), the disclosure of which is incorporated herein by reference) was used to calculate prehepatic insulin secretion from plasma C-peptide measurements with adjustment for age, sex, and BMI (see E. Van Cauter, et al., Diabetes 41, 368-377 (1992), the disclosure of which is incorporated herein by reference). The ISEC software can be obtained from the author Roman Hovorka, PhD, Metabolic Modelling Group, Centre for Measurement and Information in Medicine, Department of System Science, City University, Northampton Square, London EC1V OHB, United Kingdom. An error coefficient of variation of 5% and 15-minute intervals was required. As the program uses a population model of C-peptide kinetics, individuals were classified as diabetic (“niddm”) if they had either fasting plasma glucose >126 mg/dL, 2-hour glucose during OGTT >200, or A1C >6.5; or as “obese” if they were nondiabetic with BMI >30. The remaining individuals were classified as “normal”. Because insulin secretion is best interpreted relative to insulin resistance (insulin resistant individuals need to secrete more insulin in order to maintain normoglycemia), a disposition index (DI) was calculated, defined as the area under the insulin secretion rate, divided by the SSPG. DI thus describes beta-cell function relative to insulin resistance.

$$D I = ( \frac{{\int }_{t = 0}^{t = 30} {InsulinSecretionRate}_{t}^{OGTT}}{SSPG} )$$

Calculation—Incretin effect (IE %): Incretin effect was quantified using the equation below. Incretin effect is calculated based on the difference in the C-peptide concentrations during a frequently sampled OGTT and IIGI (0-180 min). During the IIGI, a higher incretin effect indicates an efficient incretin activity. This means normal secretion of incretin hormones (e.g., GLP-1 and GIP) and functioning binding of these hormones with the beta-cell to stimulate insulin secretion. Lower incretin effect may be due to deficiency in incretin hormones secretion or weakness in their ability to stimulate insulin secretion from the beta-cell.

$$I E \% = ( \frac{{\int }_{t = 0}^{t = 180} {cpeptide}_{t}^{OGTT} - {\int }_{t = 0}^{t = 180} {cpeptide}_{t}^{IIIGI}}{{\int }_{t = 0}^{t = 180} {cpeptide}_{t}^{OGTT}} ) * 100$$

Calculation—Hepatic IR index: Liver insulin resistance was inferred using a surrogate index as shown in the equation below, where insulin is measured in (uIU/mL or mmol/L), plasma HDL cholesterol is measured in (mg/dL or mmol/L), and BMI is measured in kg/m\hat{ }2. In adults, Body Fat % (BF %) can be estimated using the Deurenberg Index as shown below, where BMI is measured in kg/m², Age in years, and Sex=1 when male, and Sex=0 when female.

HepaticIR=−0.091+4*log(∫ₜ₌₀^{t=180} insulinₜ^{OGTT})+0.346+log(BF %)+0.408*log(HDL)+0.435*log(BMI)

BF %=−5.4+1.2*BMI+0.23*Age−10.8*Sex

Calculation— HOMA-B, HOMA-IR, and Matsuda index: Calculation were based on E. Cersosimo, et al, Curr. Diabetes Rev. 10, 2-42 (2014), the disclosure of which is incorporated herein by reference).

Identification of the dominant metabolic subphenotype: All physiological tests and calculated metabolic indicators of the participants were utilized to identify a dominant metabolic subphenotype for each person. A standardized deviance score of each metabolic subphenotype was calculated for each participant (see below), where msp(i,j) is the ith metabolic subphenotype of participant j. mean(msp(i)) is the average of j^{th} metabolic subphenotype among the cohort's participants. This measure is used as a healthy baseline for each metabolic baseline, while sd(msp(i)) is the standard deviation. High positive deviance indicates a higher risk of the corresponding metabolic phenotype, while high negative deviance indicates a healthier metabolic phenotype than the average population. Disposition index (DI) and incretin effect (IE %) were negated before applying the deviance equation, since higher DI and IE % indicate healthier beta-cell function and incretin function, respectively.

$$deviance ( {msp}_{j}^{i} ) = \frac{{msp}_{j}^{i} - mean ( {msp}^{i} )}{s d ( {msp}^{i} )}$$

where iϵ{MuscleIR, BetacellDysfunction, IncretinDysfunction, HepaticIR, AdiposeIR}

Genotyping and polygenic risk score calculation: Genotyping has been performed on the study participants using DNA extracted from PBMC. The framework is divided into five main components, namely: quality control, single nucleotide polymorphism (SNP) calling, SNP imputation using TopMed (D. Taliun, et al., Nature 590, 290-299 (2021), the disclosure of which is incorporated herein by reference), and polygenic risk score (PRS) calculations. QC, SNP calling, SNP imputation, and PRS calculations have been performed using standard protocols (S. W. Choi, et al., Nat. Protoc. 15, 2759-2772 (2020), the disclosure of which is incorporated herein by reference). PRS was calculated based on a combination of the 716 T2D-associated variants that have aggregated from three GWAS studies (M. O. Goodarzi, et al, J. Clin. Endocrinol. Metab. 105, (2020); M. Vujkovic, et al., Nat. Genet. 52, 680-691 (2020); and A. Xue, et al., Nat. Commun. 9, 2941 (2018); the disclosures of which are each incorporated herein by reference).

Machine learning framework to identify metabolic subphenotypes from OGTT glucose time series—preprocessing: Glucose time series are preprocessed by imputing missing data using linear interpolation. Then, the glucose time series are smoothed using a cubic smoothing spline with a smoothing parameter=0.35. The imputed and smoothed curves are then normalized using Z-normalization to extract features related to curve patterns rather than the amplitude.

Machine learning framework to identify metabolic subphenotypes from OGTT glucose time series—feature extraction: We extracted features for the machine learning framework using two feature extraction approaches: (a) OGTT glucose time series features (OGTT_G_Features) and (b) reduced representation of the OGTT glucose time series (OGTT_G_ReducedRep). In the first approach (OGTT_G_Features), 14 features from OGTT glucose time series were extracted, such as glucose level at time 0 (G₀), 60 (G₆₀), 120 (G₁₂₀), 180 (G₁₈₀), peak glucose level (G_Peak), length of the glucose time series (CurveSize), area under the curve (AUC), positive area under the curve (pAUC), negative area under the curve (nAUC), incremental area under the curve (iAUC), coefficient of variation (CV), time from baseline to peak value (T_baseline2peak), slope between baseline to the peak glucose level (S_baseline2peak), and slope between glucose values at the peak and at the end (at t=180 min) (S_peak2end). In the second approach (OGTT_G_ReducedRep), the OGTT glucose time series of 16 timepoints was first smoothed and Z-normalized. Then, the reduced representation of the OGTT glucose time series was extracted. This was accomplished by extracting the top two principal components of the processed OGTT glucose time series via eigen-decomposition of the covariance matrix.

Machine learning framework to identify metabolic subphenotypes from OGTT glucose time series—training: A machine learning model was developed, where the two extracted sets of glucose time series features (OGTT_G_Features and OGTT_G_ReducedRep) were tested independently. For each feature extraction approach, the dataset was split into a training set and a test set. For each metabolic subphenotype, the training was optimized for 5 different classifiers: Gaussian process classifier (GPC), support vector machine with a radial basis function kernel (SVM-RBF), support vector machine with a linear kernel (SVM-linear), logistic regression with L1 regularization (LR-L1), and logistic regression with L2 regularization (LR-L2). the python package sklearn v. ‘0.23.1’ was used to train, tune, and test machine learning models. LR-L1 and LR-L2 were trained using a SAGA solver and ran for a maximum of 1000 iterations to converge, with a regularization strength of C=10. SVM-RBF and SVM-Linear, were trained the sklearn.svm.SVC method, with kernel “linear” and “rbf”, respectively. The regularization parameter “C” for both SVM models (linear and RBF) and gamma parameter for SVM-RBF were optimized using grid search. GPC were trained using the RBF kernel, where the distance parameter and the regularization parameter “C” were tuned using grid search. The training was performed in a 4-fold cross-validation fashion and repeated 100 times, where data were shuffled before each iteration. The class imbalance problem was handled via stratified sampling to ensure that training and testing contained the same proportion of class samples. For each model, hyperparameters were tuned using grid search for each tunable hyperparameter. Importantly, in the second approach of feature extraction (OGTT_G_ReducedRep), reduced representation was obtained from the training set, not the whole dataset, and used to train each classifier. The test set was then projected onto the reduced representation space obtained from the trained dataset, and their representations were used to predict metabolic subphenotypes. Obtaining reduced representation space from the training dataset instead of the whole dataset was crucial for the robustness and generalizability of the model by preventing data leakage between the training dataset and the test dataset.

A comprehensive experiment was conducted to predict metabolic subphenotypes using the two sets of extracted features as previously discussed (OGTT_G_Features and OGTT_G_ReducedRep). Besides the extracted two sets of features from the OGTT curves, we also calculated 6 gold standard feature sets that are usually used in the clinic to infer metabolic subphenotypes, namely (1) age, sex, BMI, ethnicity, and participant family history for T2D (collectively called “Demographics” in this work), (2) demographics, A1C, and FPG (referred to “Dempgraphics+Lab” in this work), (3) HOMA-B, (4) HOMA-IR, (5) Matsuda Index, (6) total GIP and GLP-1 concentration at OGTT_2h (collectively called “Incretins” in this work). For each feature set, 5 independent classifiers (GPC, SVM-RBF, SVM-linear, LR-R1, LR-R2) were trained for each metabolic subphenotype. All tested methods and features sets were benchmarked in an exhaustive manner to test the generalizability and applicability of the use of the reduced representation of OGTT glucose time series (OGTT_G_Latent) to predict metabolic subphenotypes. Performance of each model, on each feature set, on each metabolic subphenotype was evaluated using the area under receiver operating characteristic curve (auROC).

Inferring the relationship between glucose dysregulation indicator and the metabolic subphenotypes: To assess the relative association of each metabolic phenotype to glucose dysregulation, multiple linear regression was performed of 12 combinations of 3 glucose dysregulation indicators (HbA1c, FPG, and OGTT_2h) as the dependent variables and 4 metabolic subphenotypes (muscle IR, measured in SSPG, beta-cell function measured in DI, incretin effect measured in IE, and hepatic IR measured in hepaticIndex) as predictors, while also adjusting for BMI, age, sex, and T2D genetic predisposition. Continuous predictors were mean-centered and scaled to 1 standard deviation.

Glucoselndicator_{i,j}=MSPⱼ+BMI+Age+Sex+PRS_{T2D}+ϵ_{i,j} (6)
where iϵ{HbA1c, FPG, OGTT₂ₕ} and jϵ{SSPG, DI, IE, HepaticIndex}

### Data and Results

### Unique Glycemic Responses to Oral Glucose Load

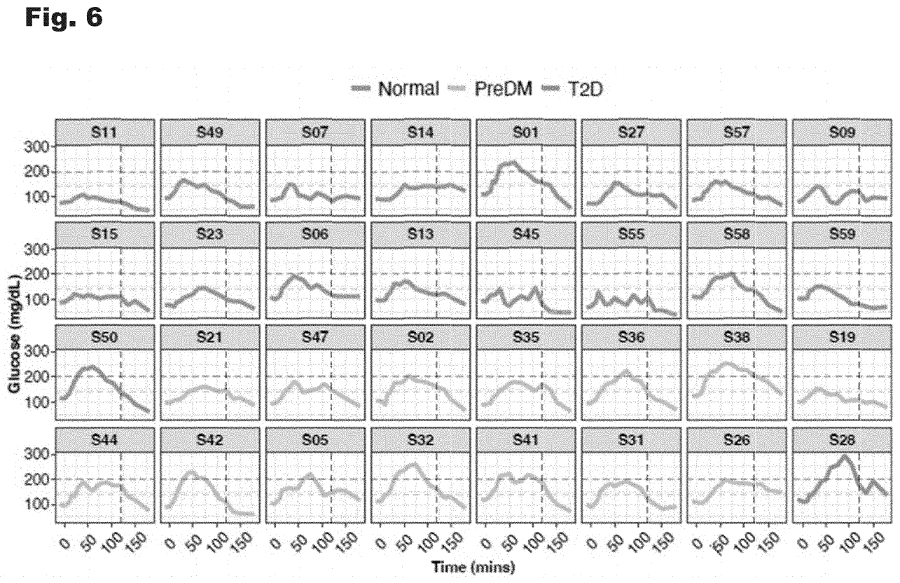

Each participant was deeply profiled throughout the OGTT tests by measuring glucose levels at 16 timepoints over three hours to generate a detailed response curve. Even though many participants fell within the same diabetes status category based on their 2-hour OGTT plasma glucose concentration (normal: glucose<140 mg/dL, prediabetes: 140≤glucose <200 mg/dL, diabetes: glucose ≥200 mg/dL), their OGTT glucose time series curves were very heterogeneous across individuals (FIG. 6). The shape of the curve was remarkably different with regard to slope, peak height attained, number of peaks, among other characteristics. Some participants exhibited large rapid glucose spikes (see S28, S42, and S01), whereas others demonstrated little excursion at all (S21, S23). Some exhibited multiple glucose spikes (S42, S47, S55) whereas others had only a single spike (S32). Additional differences are also apparent.

### Heterogeneous Responses to Metabolic Testing

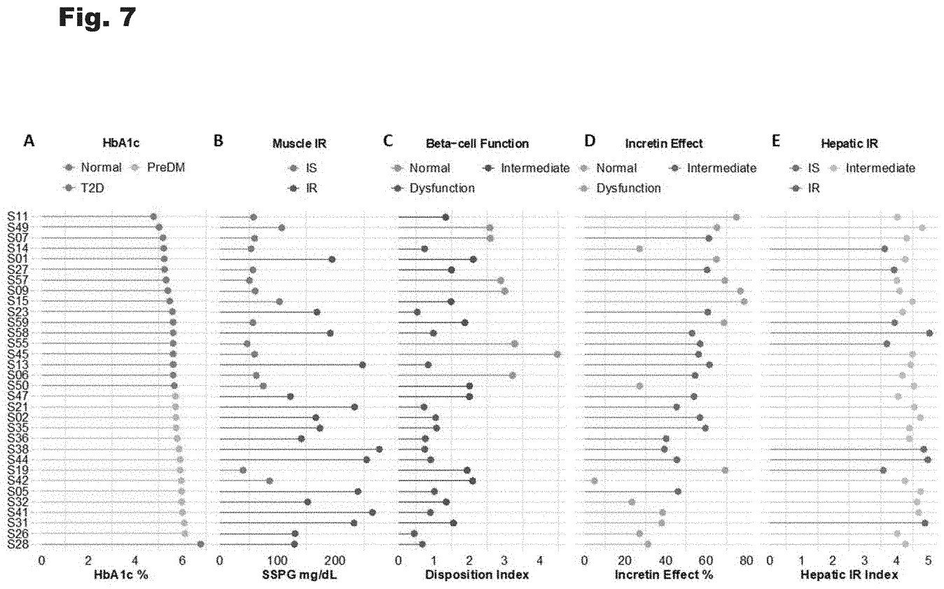

Insulin resistance, as measured by the modified insulin-suppression test and expressed as steady-state plasma glucose (SSPG), was quantified for all subjects as a continuous measure: participants were categorized as insulin sensitive (IS) if SSPG was <120 mg/dL and insulin resistant (IR) if their SSPG was >120 mg/dL. This delineation of IR encompasses individuals above the 50% of the distribution of SSPG among 490 healthy volunteers as previously published, and includes those with “moderate” elevations in SSPG who are more accurately defined as “non-IS”. This value also represented a natural cut-point in the distribution pattern of the study cohort. SSPG values spanned a wide range from 40 (subject S19) to 278 mg/dL (subject S38) (Panel A of FIG. 7). Importantly, SSPG values indicating IR (>120 mg/dL) or IS (<120 mg/dL) varied despite glycemic status—for example, some participants with elevated (prediabetes range) HbA1c, had low SSPG values indicating insulin sensitivity, whereas others with normoglycemia were quite insulin resistant.

Insulin secretion rate was calculated using the C-peptide deconvolution method with C-peptide concentrations obtained at seven timepoints (0, 15, 30, 60, 90, 120, 180 min) during the OGTT, which was then adjusted for insulin resistance to generate a measure of beta-cell function, referred to as disposition index (DI). FIG. 8 demonstrates the heterogeneity of DI in the participants at multiple time intervals during the OGTT. Participants S45 and S57 had a high DI at early time intervals following drinking the 75 g oral glucose challenge, which demonstrates a normal “healthy” beta-cell function. On the other hand, participants S28 and S58 had extremely weak DI following the oral glucose load, which indicates dysfunctional beta-cells. Beta-cell function did not necessarily relate to the glycemic status or IR/IS status (see Panels B and C of FIG. 2). For example, participant S14 was normoglycemic and IS, despite having a low DI.

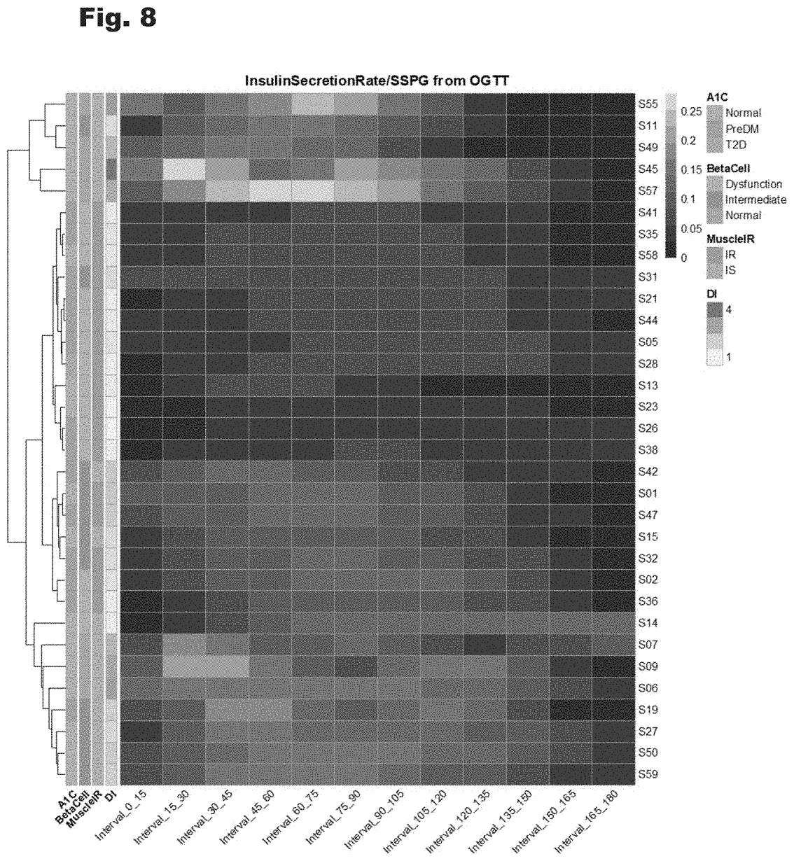

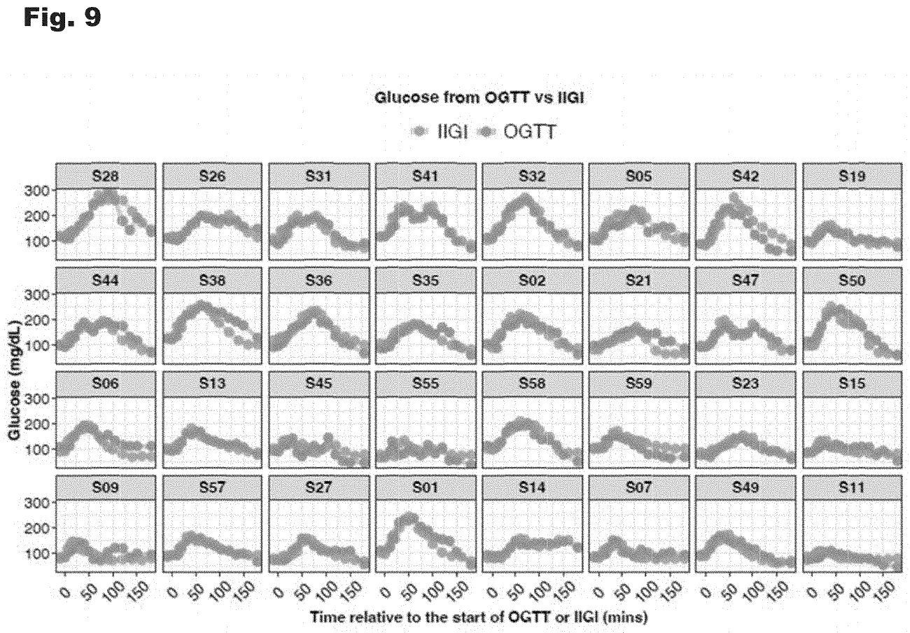

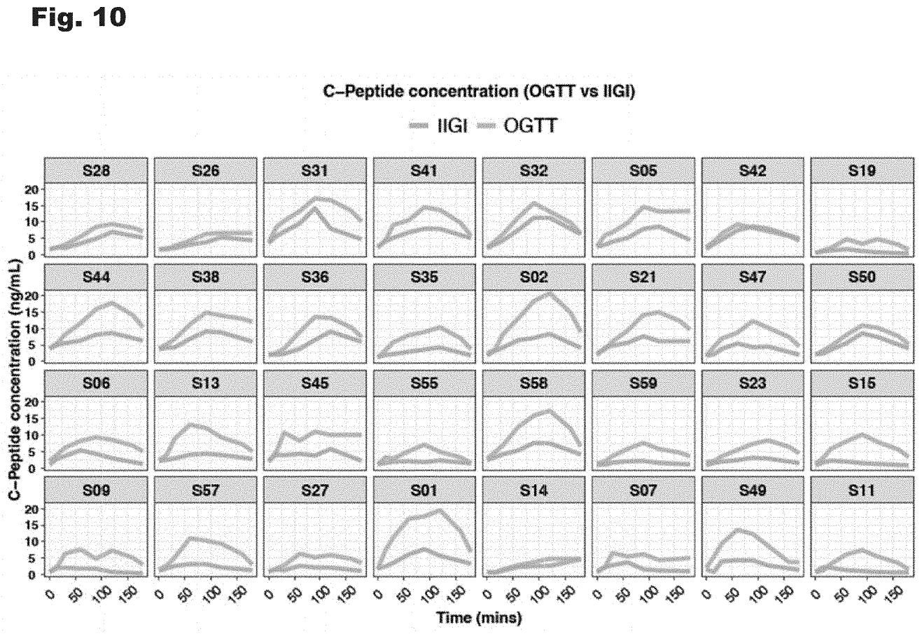

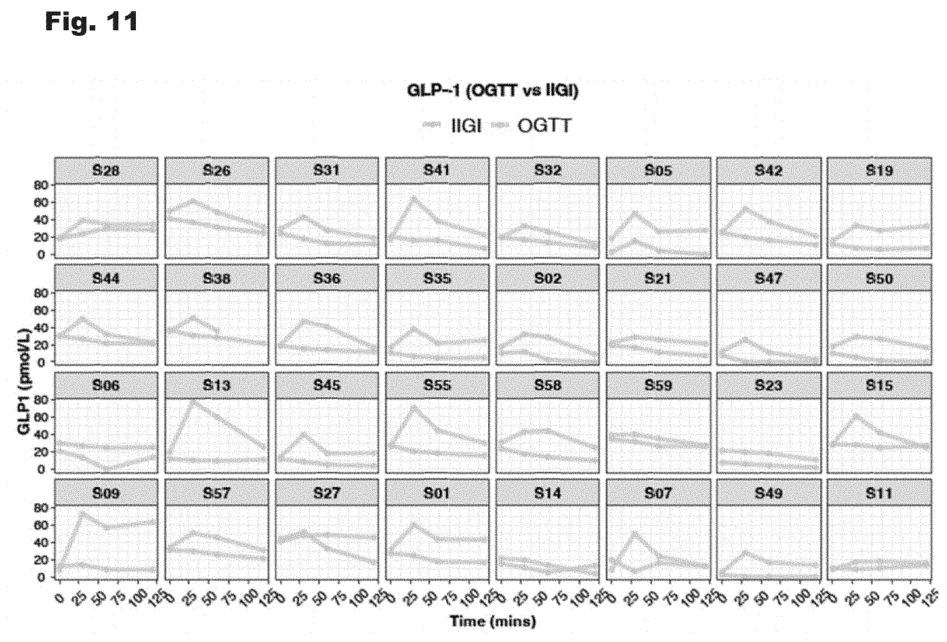

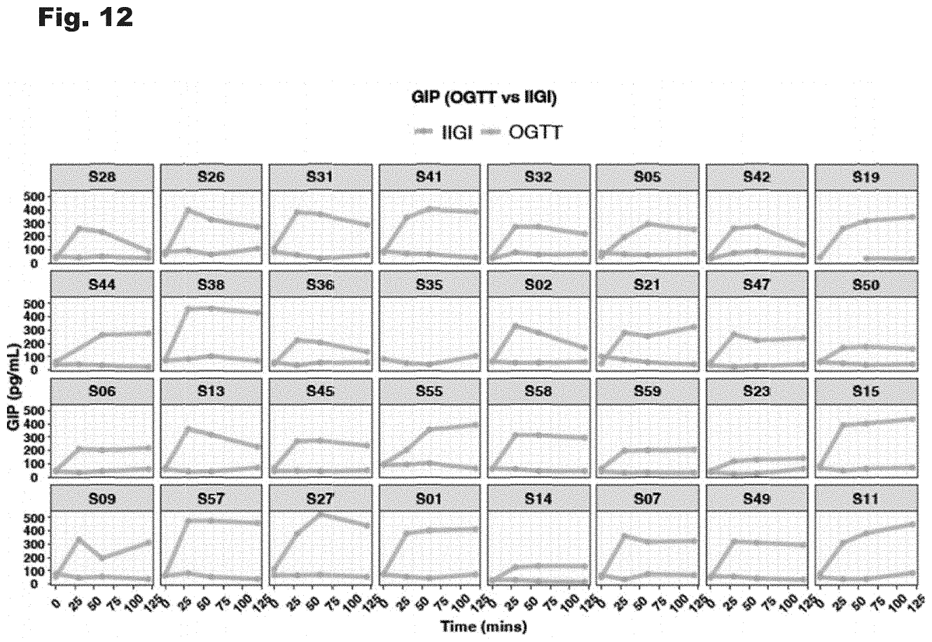

The incretin effect (IE) was measured using the gold-standard isoglycemic intravenous glucose infusion test (IIGI) in which glucose is infused intravenously to mirror the glucose curve generated after oral glucose loading (see FIG. 9 for concordance of glucose concentration between OGTT and IIGI). The difference in C-peptide concentrations during the OGTT relative to the IIGI was quantified at 4 time points (0, 30, 60, 120 min) to reflect the period of maximal hyperglycemia. The degree to which C-peptide concentrations are elevated following oral versus intravenous glucose loading reflects the incretin effect. The incretin response was extremely heterogeneous between participants. FIG. 10 depicts the C-peptide concentrations during oral (OGTT) as compared to intravenous (IIGI) glucose loading. For example, participants S42 and S32 had little difference in C-peptide responses during the OGTT and IIGI tests, indicating poor incretin effect, whereas participants S01 and S15 demonstrated a robust increase in C-peptide during OGTT versus IIGI, consistent with a high incretin effect. Of note, the secretion of incretin hormones, as measured by GLP-1, GIP, and glucagon concentrations, also varied tremendously between participants (FIGS. 11, 12, and 13, respectively).

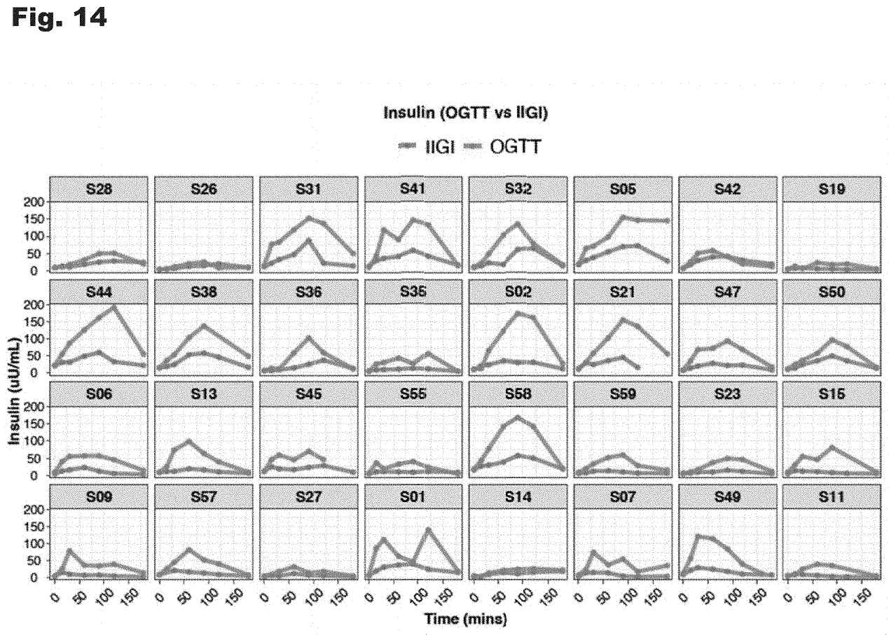

Hepatic insulin resistance (Hepatic IR) was calculated using the hepatic IR index (FIG. 14). In this work, hepatic IR index <3.95 (25th quartile in the cohort) indicates hepatic insulin sensitivity, whereas hepatic IR index>4.8 (75th quartile in our cohort) indicates hepatic IR, and a hepatic IR between 3.95 and 4.8 indicates intermediate hepatic IR. Participants S58 and S44 had the highest hepatic IR with a hepatic IR index of 5.03 and 4.98, respectively, whereas participants S19 and S14 had the lowest hepatic IR with hepatic IR of 3.57 and 3.61, respectively.

### Dominant Metabolic Subphenotype Calculation

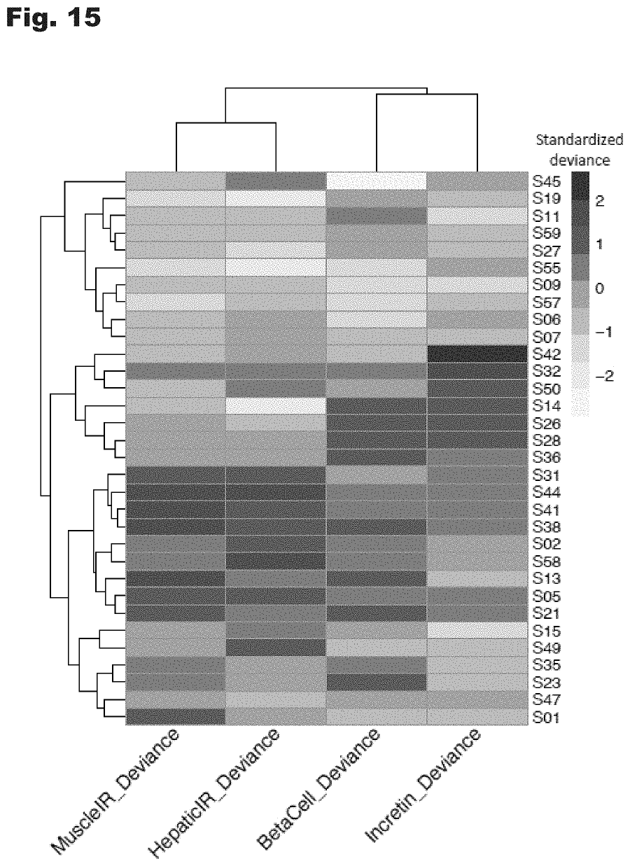

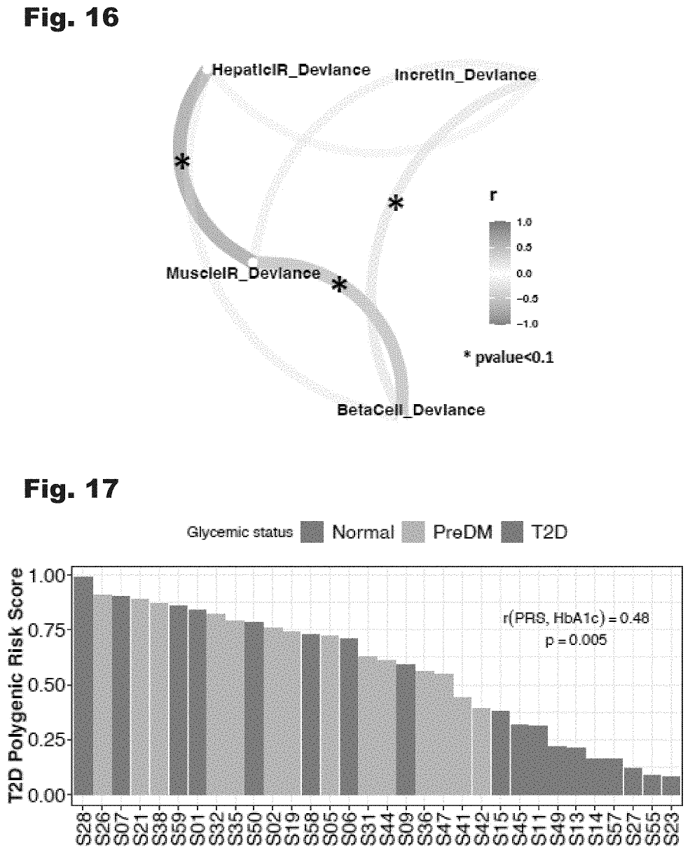

For each participant, the standard deviation values of each metabolic subphenotype was calculated as the deviation from a healthy baseline value. FIG. 15 highlights the differential deviance of each metabolic subphenotype per individual in the cohort. For each participant, the metabolic subphenotype with the highest deviance (darker red) is the metabolic subphenotype that is more adversely affected relative to other participants. For example, for participant S42, the incretin effect was the metabolic subphenotype that was most negatively affected, and for participant S23, beta-cell function was the most negatively affected metabolic subphenotype. Each individual had a different spectrum of subphenotypes demonstrating the high heterogeneity across individuals. Some individuals had only one deviant subphenotype, whereas others had several. At a cohort level, it was observed that individuals with high levels of muscle IR also had high levels of hepatic IR (r=0.73, p-value=10⁻⁵) and beta-cell dysfunction (r=0.6, p-value=2×10⁻⁴), but not impaired incretin effect (p-value=0.17 and r=0.21) (FIG. 16). Interestingly, individuals with impaired incretin effect showed a trend towards a higher level of beta-cell dysfunction (r=0.33, p-value<0.06), but not muscle or hepatic insulin resistance (r=0.25, p-value=0.17 and r=0.21, p-value=0.24, respectively). These data suggest that there are distinct mechanisms for each metabolic subphenotype, that great heterogeneity exists between individuals for each subphenotype, and that a dominant metabolic subphenotype can be determined for each individual.

### Polygenic Risk Score as Predictor of Glycemic Level

Each participant was genotyped using a genome-wide array and the genetic predisposition for T2D quantified by calculating the T2D polygenic risk score (PRS_{T2D}) based on >700 independent signals for T2D-risk. FIG. 17 shows that the PRS_{T2D} is associated with HbA1c level (r=0.48, p-value=0.005). A notable exception is female participant S07 (age at time of study 37 yrs, BMI 26.3 kg/m²) who had a high PRS_{T2D} of 0.9, despite normal muscle insulin sensitivity and beta-cell function (SSPG=60 mg/dL, DI=2.58, Panels B and C of FIG. 7, respectively), and intermediate incretin effect and hepatic insulin resistance (IE=63.7%, hepatic IR index=4.3, Panels D and E of FIG. 7, respectively). As expected, PRS_{T2D} was positively correlated, but did not reach significance level, with SSPG (r=0.26, p-value=0.14), hepatic IR (r=0.28, p-value=0.1), HOMA-IR (r=0.3, p-value=0.08), BMI (r=0.18, p-value=0.29), and Triglycerides (r=0.22, p-value=0.21). Also, PRS_{T2D} was inversely correlated, but did not reach significant levels, with DI (r=−0.2, p-value=0.25), IE (r=−0.16, p-value=0.36), HOMA-B (r=−0.32, p-value=0.06), and HDL (r=−0.28, p-value=0.12).

### Features of the Glucose Response Curve During OGTT Identify and Predict Metabolic Subphenotypes

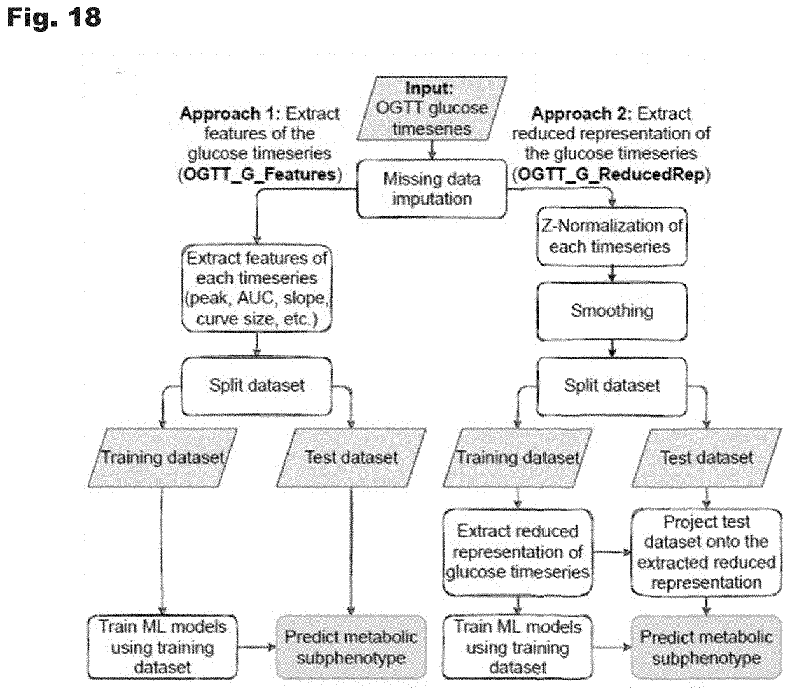

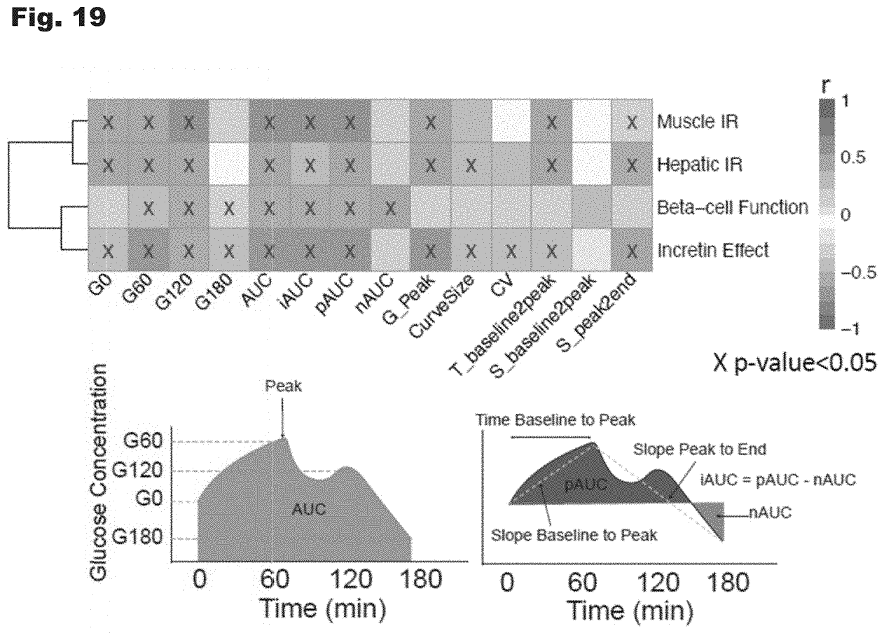

To determine if the metabolic subphenotypes could be predicted from the glucose time series response to the standardized OGTT test a robust machine learning framework was developed. Features from the OGTT glucose time series were extracted using two approaches: (a) engineered features from the OGTT glucose time series (OGTT_G_Features) (b) a reduced representation method (OGTT_G_ReducedRep) that represents each smoothed and normalized OGTT curve with a vector of 2 dimensions (FIG. 18). In the first approach (OGTT_G_Features), 14 features were extracted from the OGTT glucose time series. Multiple extracted features from OGTT glucose time series were significantly correlated with metabolic subphenotypes (p-value<0.05, marked as x on FIG. 19). AUC and iAUC were the top two significantly positively correlated features with muscle IR (r=0.59 and p-value=3×10⁻⁴, r=0.57 and p-value=6×10⁻⁴, respectively). G₁₂₀ and iAUC were the top two negatively correlated with beta-cell function (r=−0.59 and p-value=4×10⁻⁴, r=−0.55 and p-value=3×10⁻³, respectively). pAUC and G_Peak were the top two features significantly negatively correlated with the incretin effect (r=−0.72 and p-value=6×10⁻⁵, r=−0.68 and p-value=2×10⁻⁵, respectively). Finally, S_peak2end and G₀ were the top two parameters significantly correlated with the hepatic IR (r=−0.51 and p-value=0.002, r=0.47 and p-value=0.006, respectively).

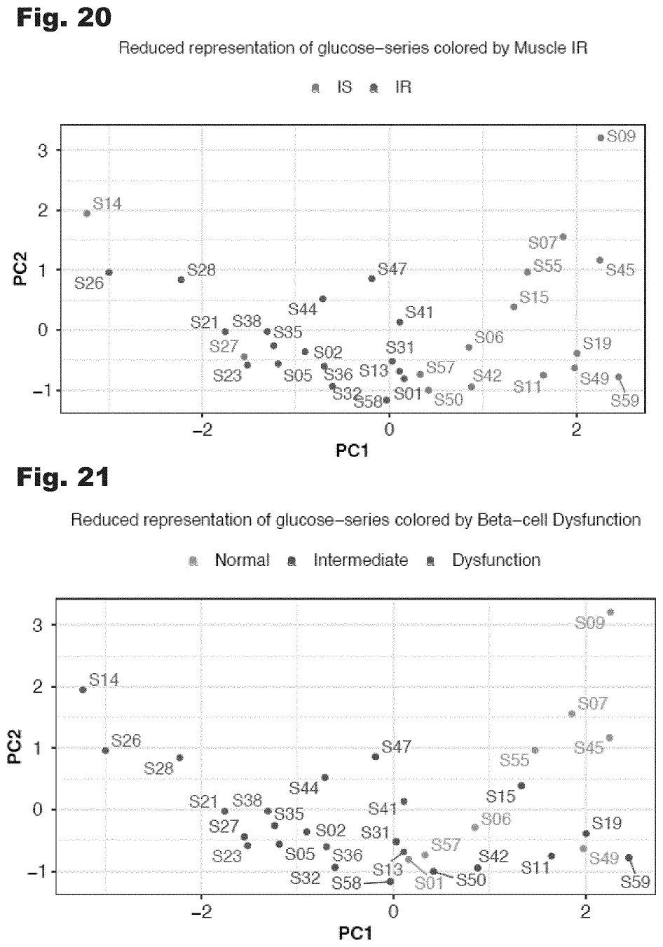

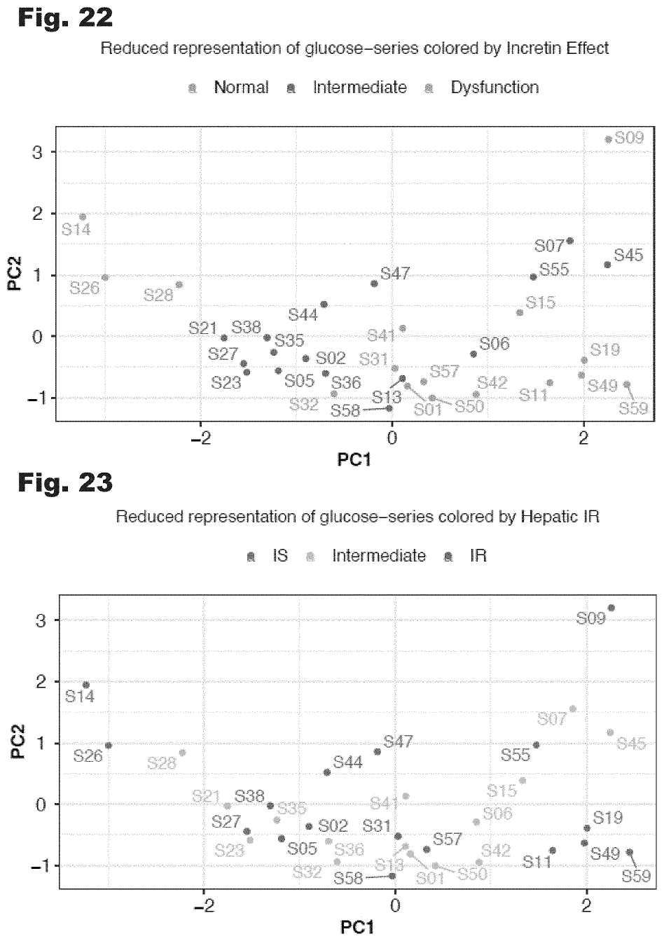

In the second approach (OGTT_G_ReducedRep), the OGTT glucose time series of 16 timepoints was smoothed and Z-normalized. Then, the reduced representation of the OGTT glucose time series was extracted. Interestingly, using the reduced representation of the OGTT glucose time series, there was a very clear separation of muscle IR and IS (FIG. 20). Additionally, the reduced representation of OGTT glucose time series readily distinguished the three classes for beta-cell (normal, intermediate, and dysfunction) (FIG. 21). Individuals with normal beta-cell function were located on the positive side of the first principal component (PC1), whereas individuals with beta-cell dysfunction were found on the negative side of PC1, and individuals with intermediate beta-cell function are found in between. The same pattern held true for incretin effect classes (FIG. 22), where participants with normal versus impaired incretin effect were reasonably well separated, whereas participants with intermediate incretin effect overlapped with the two more extreme groups. On the other hand, hepatic IR classes were not separable using the reduced representation of the processed OGTT glucose time series (FIG. 23).

Predicting Metabolic Subphenotypes from Glucose Time Series Using Machine Learning

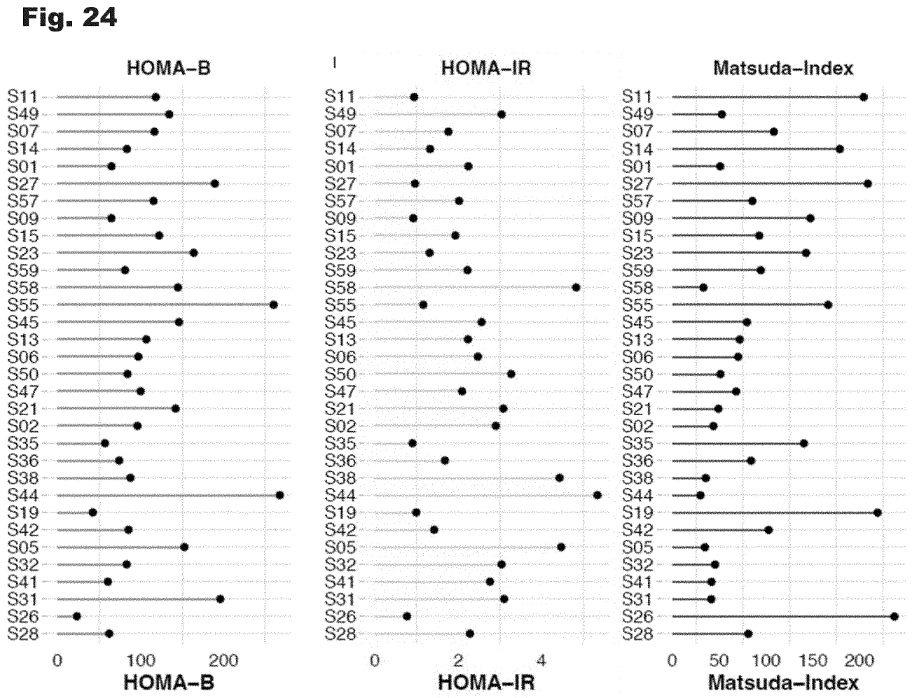

The separability in the classes of muscle insulin sensitivity, beta-cell function, and incretin effect, using the reduced representation of the processed OGTT glucose time series suggested that a robust prediction of such classes could be achieved using a learning framework. For each feature extraction approach, the dataset was split into a training set and a test set. For each metabolic subphenotype, the training was optimized trying five different classifiers: Gaussian process classifier (GPC), support vector machine with a radial basis function kernel (SVM-RBC), support vector machine with a linear kernel (SVM-linear), logistic regression with L1 regularization (LR-L1), and logistic regression with L2 regularization (LR-L2). Models were evaluated comprehensively to ensure generalizability and robustness of the trained models. Prediction of metabolic subphenotypes was benchmarked using the two sets of extracted features from the OGTT timeseries with surrogate metabolic measures in current use including: (1) Demographics (age, sex, BMI, ethnicity, and participant family history for T2D); (2) Demographics+Lab (demographic variables plus A1C, and FPG); (3) HOMA-B, a surrogate marker for beta-cell function; (4) HOMA-IR and (5) Matsuda Index, surrogate markers for muscle IR; and (6) total GIP and GLP-1 concentrations at OGTT_2h (an optimized surrogate marker for incretin effect) (FIG. 24). Performance of each model, on each feature set, on each metabolic subphenotype was evaluated using the area under the receiver operating characteristic curve (auROC). Statistical significance is performed between the measure of auROCs among all tested features and OGTT_G_ReducedRep using the Wilcoxon Rank Sum Test.

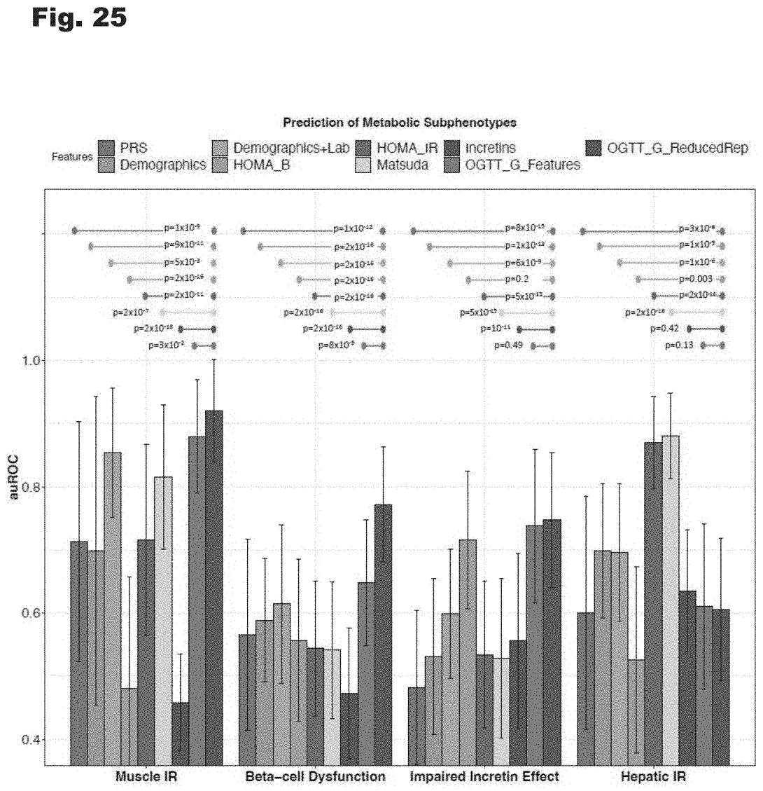

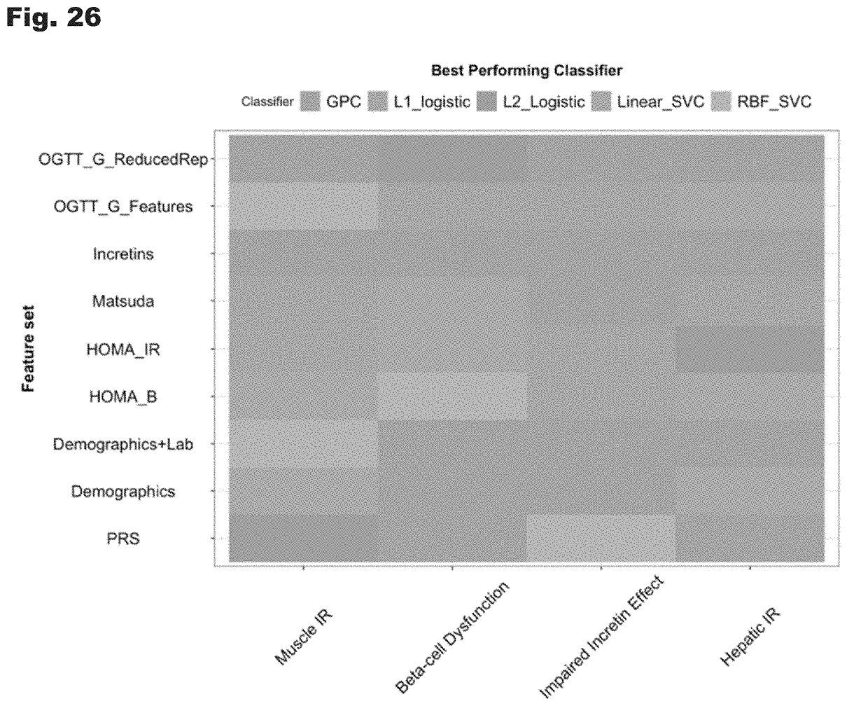

FIGS. 25 and 26 summarizes the auROC of the best performer classifier for metabolic subphenotype using each feature set. Muscle IR was predicted with extraordinarily high auROC=0.94 using OGTT_G_ReducedRep via GPC. OGTT_G_Feature predicted Muscle IR with auROC=0.88. OGTT_G_ReducedRep had significantly higher predictive power than any one of the currently used features to predict Muscle IR, including the optimized measure of Demographics+Lab (auROC=0.85, p-value=5×10⁻³), HOMA-IR (auROC=0.71, p-value=2×10⁻¹¹), and Matsuda index (auROC=0.81, p-value=2×10⁻⁷). Beta-cell dysfunction was predicted from OGTT_G_ReducedRep with an auROC of 0.76 via logistic regression with L2 regularization (LR-L2), which is significantly higher than Demographics+Lab (auROC=0.61, p-value=2×10⁻¹⁶) and HOMA-B (auROC=0.57, p-value=2×10⁻¹⁶). Incretin effect class could be predicted with an auROC of 0.75 using the OGTT_G_ReducedRep via GPC, which was higher than the top performer feature set of the current tests, which was HOMA-B (auROC=0.71, p-value=0.2). Surprisingly, incretin hormones, widely used surrogate markers for the incretin effect, showed poor prediction of the incretin effect compared to OGTT_G_ReducedRep (auROC=0.56, p-value=10⁻¹¹). OGTT_G_ReducedRep was not superior to current surrogate estimates and showed a lower predictive power in predicting Hepatic IR (auROC=0.65) compared to the Matsuda Index (auROC=0.88, p-value=2×10⁻¹⁶), or HOMA-IR (auROC=0.87, p-value=2×10⁻¹⁶).

### Relationship Between Incretin Effect and Incretin Hormones

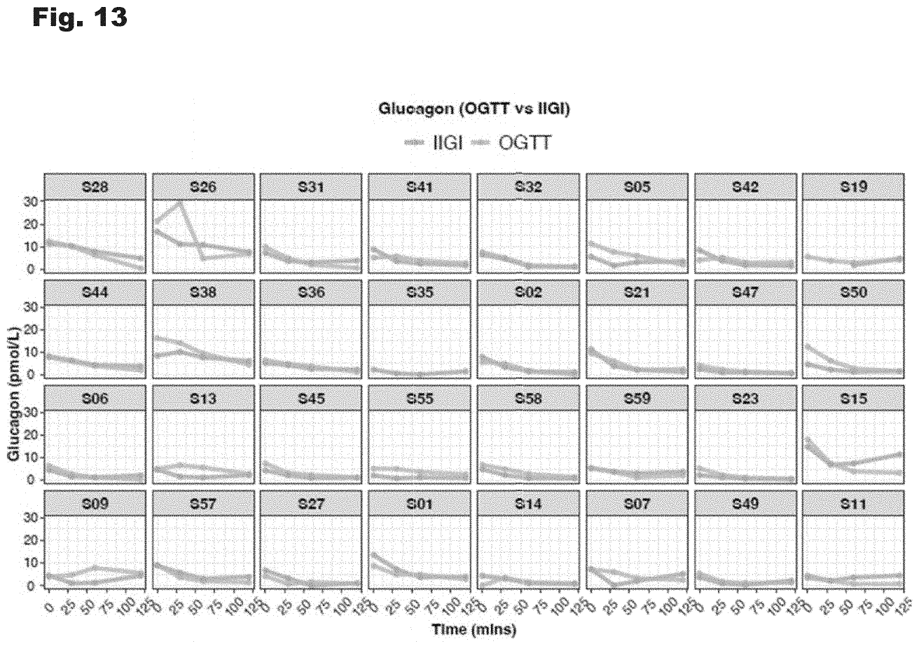

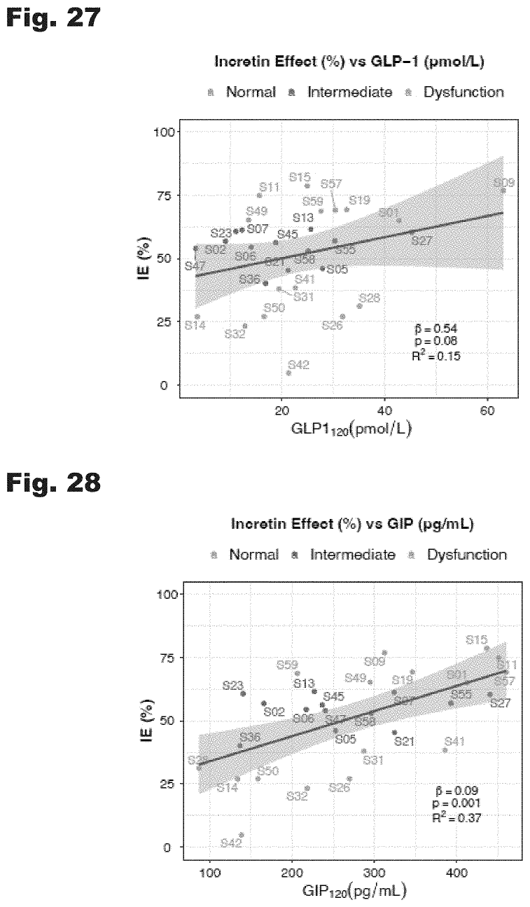

To better understand the relationship between the incretin effect and incretin hormone levels, the gold-standard measure of incretin effect was compared with the 2-hour plasma concentrations of GLP-1 and GIP (FIG. 13). Notably, concentrations of peak (120 min following glucose load) GLP-1, often used as a surrogate marker for incretin response, did not correlate (BMI and age-adjusted) with the gold-standard measure incretin effect as described above (FIG. 27). On the other hand, peak GIP correlated modestly but significantly with quantitative incretin effect (p-value=0.001, FIG. 28). The lack of a robust correlation between incretin hormone concentrations and incretin effect is exemplified in participant S41, who had moderate levels of GLP-1 (22.6 pmol/L) and a high level of GIP (385 μg/mL) at 120 min but a low incretin effect (38%).

### Relationship Between Metabolic Subphenotypes and Glucose Dysregulation Indicators

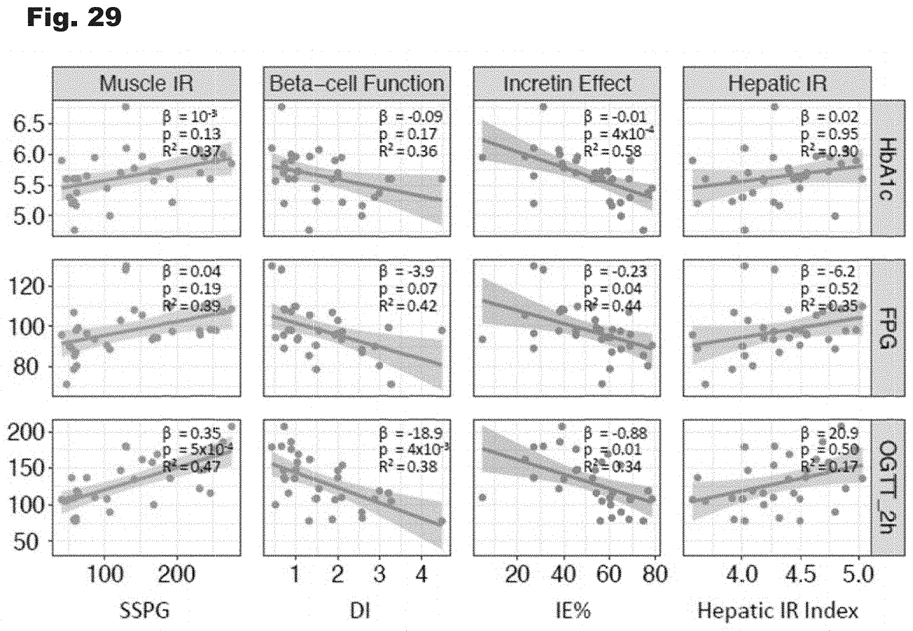

To assess the relative association of each metabolic phenotype to common measurements of glucose dysregulation, multiple linear regression was performed on 12 combinations of three common glucose dysregulation indicators (HbA1c, FPG, and OGTT_2h) as the dependent variables and four metabolic subphenotypes (muscle IR, expressed as SSPG, beta-cell function expressed as DI, incretin effect expressed as IE, and hepatic IR expressed as Hepatic IR Index) as predictors, while also adjusting for BMI, age, sex, and T2D genetic predisposition. Continuous predictors were mean-centered and scaled to one standard deviation. As shown in FIG. 29, among the four metabolic subphenotypes quantified by gold-standard tests, only the incretin effect was significantly associated with the three glucose dysregulation indicators: HbA1c (β=−0.01, R²=0.58, p-value=4×10⁻⁴), FPG (β=−0.23, R²=0.44, p-value=0.04), and OGTT_2h (β=−0.88, R²=0.34, p-value=0.01). Beta-cell function was significantly associated with OGTT_2h (β=−18.9, R²=0.38, p-value=0.004). Muscle IR was significantly associated with OGTT_2h (β=0.35, R²=0.47, p-value=5×10⁻⁴). Calculated Hepatic IR was not significantly associated with any of the three glucose indicators. As expected, BMI, age, T2D polygenic risk score, and males had a positive association with the three glucose indicators, although not statistically significant. In total, common indicators of glucose dysregulation were not strong predictors of metabolic subphenotypes.

### Discussion

Here the data and results demonstrate that individuals with glucose ranging from normal to prediabetes exhibit clear heterogeneity in four distinct physiologic processes that contribute to disordered glucose metabolism, including muscle IR, beta-cell dysfunction, impaired incretin effect, and hepatic IR. By performing deep metabolic phenotyping with gold-standard tests, the results show that one or another of these defects may predominate in a given individual and most individuals exhibit a dominant metabolic subphenotype. T2D and its precursor, prediabetes, are currently defined solely on the basis of glycemic level rather than underlying pathology. The mechanistic basis, however, is a complex interplay between genetic, epigenetic, proteomic and metabolomic processes in multiple tissues (muscle, adipose, liver, and intestine). Historically, subclassification of T2D into more specific subtypes has been limited by incomplete understanding of the physiology and inability to identify the specific subtypes using reasonably available and accurate diagnostic tests.

Just as separating type 1 and T2D has critically important implications for treatment, identifying subtypes within the T2D and even prediabetes category has important treatment implications, particularly as treatments now exist that target each separate pathogenic mechanism, with metformin targeting hepatic IR, GLP-1 analogs and DPP4 inhibitors targeting incretin deficiency, thiazolidenediones targeting muscle IR, and sulfonylureas, glinides and insulin targeting beta-cell deficiency. The benefit of subtyping T2D according to underlying pathology likely extends to prediabetes as well, given that the same medications used to treat T2D have also been shown to prevent conversion to T2D by up to 79% in high-risk individuals with prediabetes, who have a 70% chance of progressing to overt T2D.

Furthermore, among normoglycemic overweight and obese individuals who are IR, dietary weight loss significantly improves cardiometabolic risk profile. Equally-obese individuals who are not IR do not show a similar metabolic benefit, indicating that if it were possible to identify IR at baseline, weight loss efforts could be targeted towards the IR subset of overweight/obese individuals, which is estimated to represent over 40% of the US adult population. In addition to identifying the IR subset of normoglycemic overweight/obese who will benefit from lifestyle efforts to prevent T2D, deeper metabolic subphenotyping may have implications for the targeted amount of weight loss. For example, 5% weight loss has a modest effect on beta-cell function as well as insulin sensitivity, whereas progressive weight loss beyond 5% further impacts only insulin sensitivity. Dietary macronutrients also differentially affect the specific underlying pathophysiologic contributors to T2D. metabolic of hyperglycemia. For example, substitution of dietary monounsaturated for saturated fat reduces muscle IR but does not improve insulin secretion. Alternatively, while diets low in saturated fats as compared to unsaturated fats and simple sugars decreases de novo lipogenesis and hepatic IR, some studies show that total fat reduction is more important and that unsaturated fats can also induce hepatic IR. In relation to incretin action, a number of studies point to exciting specific dietary benefits of whey, olive oil, dietary fiber, and caffeine.

Although the current understanding of the pathology underlying T2D as well as the range of effective treatment strategies has expanded tremendously, the inability to practically subphenotype individuals according to the underlying physiology has hindered the development of a more detailed classification system and targeted treatment/prevention. The results demonstrate that distinct metabolic subphenotypes are quantifiable using features derived from a standard OGTT with frequent blood sampling. A robust machine learning framework was employed that utilizes 16 plasma glucose values obtained every 5-30 minutes following oral glucose ingestion. The frequency of measurements provided a curve shape at high resolution that was greater in predictive value than level of glycemia, clinical-demographic profile, polygenic risk score, or currently used surrogate markers to identify muscle insulin resistance, beta-cell function, or incretin effect. Furthermore, unlike other surrogate tests which aim to identify a single metabolic subtype, this single test accurately identifies three important metabolic subtypes, each of which is amenable to targeted lifestyle and pharmacologic therapy as described. As such, the described approach has the potential to subphenotype T2D in its earliest clinical stages, prior to overt hyperglycemia, setting the stage for a new classification approach based on the underlying pathology. The best overall measure was based on the reduced representation of the processed glucose time series (OGTT_G_ReducedRep). Gold standard tests to identify muscle IR “insulin suppression test”, takes more than 6-hours to be performed and requires a professional, which makes it expensive and necessitates a clinical setting. Similarly, measuring beta-cell function requires measuring C-peptide at different time intervals during the OGTT which is not standardized between labs. Incretin effect calculations require a 4 hr IIGI test during which C-peptide is measured at different time points. Altogether identifying metabolic subphenotypes using current gold-standard methods is impractical to perform on a large scale.

Compared to the alternative of gold-standard metabolic tests, which are labor-intensive, time-consuming, costly, and must be performed in a clinical research unit, the described methods are significantly more feasible, and the benefit may be viewed to outweigh the burden of inconvenience. Finally, these methods yield predictive accuracy that surpasses currently available measures such as clinical-demographic, level of glycemia, polygenic risk score, and surrogate markers such as HOMA and Matsuda index. Thus, high risk individuals might consider utilizing this test in the clinical laboratory or ambulatory care unit and could be ordered by their physicians. Moreover, the current results provide a proof of principle for prediction of metabolic subphenotypes using continuous glucose monitoring devices.

In summary, the data and results demonstrate that the metabolic underpinnings of T2D vary widely between individuals and that dominant metabolic subphenotypes exist. It was further shown that modeling a glucose time series response to oral glucose challenge can identify these metabolic subphenotypes. The features of the glucose time series identified muscle IR, beta-cell deficiency, and incretin deficiency with superior predictive power than standard clinical and laboratory measures, accepted plasma surrogate markers, and T2D polygenic risk score. Given the known variability in clinical response to virtually all antidiabetic medications and the increasing availability of choices that target different physiologic pathways to hyperglycemia, the potential for metabolic subphentoyping in T2D to impact health outcomes and cost savings is significant. As such, the current results provide support for a new subclassification approach to T2D and prediabetes that considers the underlying pathology and has potential to improve health outcomes and expenditures.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A method to assess an underlying pathology of metabolic dysregulation in an individual, comprising:
obtaining data results of a glucose response curve that is generated from an individual;
generating features from the data results of the glucose response curve; and
assessing, utilizing a trained computational model, an underlying pathology of metabolic dysregulation, wherein the trained computational model is trained to predict an indicator of the underlying pathology.

2. The method of claim 1, wherein the underlying pathology is muscular insulin resistance, beta cell dysfunction, impaired incretin effect, or hepatic insulin resistance.

3. The method of claim 1, wherein the glucose response curve is obtained from a continuous glucose monitor (CGM).

4. The method of claim 1, wherein the glucose response curve is obtained from an oral glucose tolerance test (OGTT) or an assessment involving administration of a glucose load.

5. The method of claim 1, wherein the features are extracted from the data results of the glucose response curve.

6. The method of claim 5, wherein the extracted features comprises at least one of the following: glucose level at 0 seconds (G₀), glucose level at 60 minutes (G₆₀), glucose level at 120 minutes (G₁₂₀), glucose level at 180 minutes (G₁₀), peak glucose level (G_Peak), length of the glucose time series (CurveSize), area under the curve (AUC), positive area under the curve (pAUC), negative area under the curve (nAUC), incremental area under the curve (iAUC), coefficient of variation (CV), time from baseline to peak value (T_baseline2peak), slope between baseline to the peak glucose level (S_baseline2peak), or slope between glucose values at the peak and at the end (at t=180 min) (S_peak2end).

7. The method of claim 1 further comprising generating a reduced representation of the data results of the glucose response curve.

8. The method of claim 7, wherein generating a reduced representation comprises smoothing and Z-normalizing the data results of the glucose response curve.

9. The method of claim 8 further comprising extracting one or more top principal components via eigen-decomposition of a covariance matrix of the smoothed and Z-normalized data results of the glucose response curve.

10. The method of claim 1, wherein the computational model is one of the following: a Gaussian process classifier (GPC), a support vector machine with a radial basis function kernel (SVM-RBF), a support vector machine with a linear kernel (SVM-linear), a logistic regression with L1 regularization (LR-L1), or a logistic regression with L2 regularization (LR-L2).

11. The method of claim 1 further comprising determining a contribution of the underlying pathology of metabolic dysregulation based on a deviance from a healthy underlying pathology.

12. The method of claim 11, wherein the deviance from the healthy underlying pathology is determined by an average underlying pathology score from a collection of individuals.

13. The method of claim 12, wherein each individual of the collection of individuals has a similar overall metabolic assessment.

14. The method of claim 11, wherein the determination of the contribution of the underlying pathology of metabolic dysregulation based on a deviance from a healthy pathology identifies a dominant underlying pathology of metabolic dysregulation.

15. The method of claim 11, further comprising performing an additional clinical assessment based on the contribution of the underlying pathology of metabolic dysregulation.

16. The method of claim 15, wherein the additional clinical assessment is to confirm the assessment of the underlying pathology.

17. The method of claim 11 further comprising administering a treatment to the individual based on the contribution of the underlying pathology of metabolic dysregulation.

18. The method of claim 17, wherein beta cell dysregulation is determined to contribute to the metabolic dysregulation, the method further comprising administering an agent to improve insulin secretion.

19. The method of claim 17, wherein muscular insulin resistance is determined to contribute to the metabolic dysregulation, the method further comprising administering an agent to improve insulin sensitivity.

20. The method of claim 17, wherein hepatic insulin resistance is determined to contribute to the metabolic dysregulation, the method further comprising administering an agent to decrease hepatic glucose production.

21. The method of claim 17, wherein impaired incretin effect is determined to contribute to the metabolic dysregulation, the method further comprising administering a GLP-1 receptor agonists or a DPP-4 inhibitor.

22. The method of claim 17, wherein the treatment comprises one of the following: insulin, alpha-glucosidase inhibitors, biguanides, dopamine agonists, DPP-4 inhibitors, GLP-1 receptor agonists, meglitinides, sodium glucose transporter 2 inhibitors, sulfonylureas, or thiazolidinediones.

23. The method of claim 17, wherein the treatment comprises one of the following: alpha-lipoic acid, chromium, coenzyme Q10, garlic, hydroxychalcone (cinnamon), magnesium, omega-3 fatty acids, psyllium or vitamin D.

24. The method of claim 17, wherein the treatment comprises one of the following: a dietary alteration, an increase in exercise, or stress management.

25. A method for stratifying a prediabetic individual based on an underlying pathology of metabolic dysregulation, comprising:
obtaining data results of a glucose response curve that is generated from a prediabetic individual;
generating features from the data results of the glucose response curve; and
assessing, utilizing a trained computational model, an underlying pathology of metabolic dysregulation, wherein the trained computational model is trained to predict an indicator of the underlying pathology.

26. The method of claim 25, wherein the prediabetic individual has been diagnosed as prediabetic.

27. The method of claim 26, wherein the diagnosis is based upon HbA1c levels or fasting glucose levels.

28. The method of claim 25, wherein the underlying pathology is muscular insulin resistance, and wherein the indicator of the underlying pathology provides an indication of insulin resistance.

29. The method of claim 28 further comprising determining a contribution of the muscular insulin resistance based on a deviance from a healthy underlying muscular insulin resistance pathology.

30. The method of claim 29, wherein the deviance from the healthy muscular insulin resistance underlying pathology is determined by an average insulin resistance score from a collection of prediabetic individuals.

31. The method of claim 30 further comprising administering a treatment to the individual based on the contribution of the underlying pathology of metabolic muscular insulin resistance dysregulation, wherein the contribution stratifies the prediabetic individual as insulin sensitive or insulin resistant.

32. The method of claim 31, wherein the prediabetic individual is stratified as insulin resistant and the individual is administered an agent to improve insulin sensitivity along with a dietary alteration and an increase in exercise.

33. The method as in claim 32, wherein the agent to improve insulin sensitivity is a thiazolidinedione.

34. The method of claim 31, wherein the prediabetic individual is stratified as insulin sensitive and the individual is administered a dietary alteration and an increase in exercise without administering an agent to improve insulin sensitivity.
